(19) United States
(12) Patent Application Publication
(10) Pub. No.: US 2016/0151406 A1
(43) Pub. Date: Jun. 2, 2016

(54) COMBINATION CANCER THERAPY WITH C-MET INHIBITORS AND SYNTHETIC OLIGONUCLEOTIDES

(71) Applicant: Mirna Therapeutics, Inc., Austin, TX (US)

(72) Inventors: Andreas BADER, Austin, TX (US); Jane ZHAO, Austin, TX (US); Adriana GUERRERO, Austin, TX (US)

(21) Appl. No.: 14/946,456

(22) Filed: Nov. 19, 2015

Related U.S. Application Data
(60) Provisional application No. 62/081,882, filed on Nov. 19, 2014.

(51) Int. Cl. A61K 31/713; C12N 15/113; A61K 31/573; A61K 9/127; A61K 31/475; A61K 31/506

(52) U.S. Cl. CPC A61K 31/713; A61K 31/475; A61K 31/506; A61K 31/573; A61K 9/127; C12N 15/1135; C12N 2310/141; C12N 2320/31

(57) ABSTRACT

Methods of inhibiting, and preventing the proliferation of, cancer cells, as well as treating cancer in an individual (e.g., a liver cancer, for example hepatocellular carcinoma) can include providing both a synthetic miR-34 family molecule and a c-Met inhibitor (e.g., tivantinib) to an individual in need thereof. The combination of the synthetic miRNA molecule and c-Met inhibitor can provide a desirable or superior effect, for example a more efficacious treatment than an alternative therapy, or the synthetic miRNA molecule or c-Met inhibitor alone. In some embodiments, the combinations provide a synergistic or greater than additive effect, or reduce toxicity and/or other side effects.

### CROSS-REFERENCE TO RELATED APPLICATIONS

This application claims the benefit of priority to U.S. Provisional Application No. 62/081,882, filed on Nov. 19, 2014, which incorporated herein by reference in its entirety.

### SUMMARY OF THE INVENTION

Disclosed herein, in certain embodiments, are methods of treating a cancer in an individual in need thereof, comprising: administering to the individual: (a) a c-Met inhibitor; and (b) a synthetic miRNA molecule, comprising: (a) an active strand comprising a sequence at least 80% identical to a mature miRNA; and (b) a separate complementary strand that is at least 60% complementary to the active strand. In some embodiments, the passenger strand of the synthetic miRNA molecule comprises a 5′ terminal cap. In some embodiments, the 5′ terminal cap is a lower alkylamine. In some embodiments, the 5′ terminal cap is NH₂—(CH₂)₆—O—. In some embodiments, the mature miRNA molecule is miR-34a (SEQ ID NO: 1). In some embodiments, the mature miRNA molecule is miR-34b (SEQ ID NO: 2). In some embodiments, the mature miRNA molecule is miR-34c (SEQ ID NO: 3). In some embodiments, the mature miRNA molecule comprises a miR-34 consensus sequence (SEQ ID NO: 4). In some embodiments, the mature miRNA molecule is miR-449a (SEQ ID NO: 5). In some embodiments, the mature miRNA molecule is miR-449b (SEQ ID NO: 6). In some embodiments, the mature miRNA molecule is miR-449c (SEQ ID NO: 7). In some embodiments, the mature miRNA molecule comprises a miR-449 consensus sequence (SEQ ID NO: 8). In some embodiments, the mature miRNA molecule comprises a miR-34/449 seed sequence (SEQ ID NO: 9). In some embodiments, the cancer is pancreatic, gastric, lung, thyroid, brain, kidney, head and neck, or liver cancer. In some embodiments, the cancer is liver cancer. In some embodiments, the liver cancer is primary liver cancer. In some embodiments, the liver cancer is hepatocellular carcinoma (HCC). In some embodiments, the c-Met inhibitor and the synthetic miRNA molecule are administered concurrently. In some embodiments, the c-Met inhibitor and the synthetic miRNA molecule are administered sequentially. In some embodiments, the c-Met inhibitor and the synthetic miRNA molecule are administered in a unified dosage form. In some embodiments, the c-Met inhibitor and the synthetic miRNA molecule are administered in separate dosage forms. In some embodiments, the c-Met inhibitor is selected from the group consisting of: ARQ197 (Tivantinib), GSK/1363089/XL880 (Foretinib), XL184 (Cabozantinib), HMPL-504/AZD6094/volitinib (Savolitinib), MSC2156119J (EMD 1214063, Tepotinib), LY2801653 (Merestinib), AMG 337, INCB28060 (Capmatinib), AMG 458, PF-04217903, PF-02341066 (Crizotinib), E7050 (Golvatinib), MK-2461, BMS-777607, JNJ-38877605, EMD1214063 (MSC2156119J; Tepotinib), SOMG-833, or pharmaceutically acceptable salts thereof. In some embodiments, the c-Met inhibitor is an ATP non-competitive c-Met inhibitor. In some embodiments, the c-Met inhibitor is ARQ197 (tivantinib). In some embodiments, the c-Met inhibitor is EMD1214063 (MSC2156119J; Tepotinib). In some embodiments, the c-Met inhibitor and synthetic miRNA molecule are administered in molar ratio of about 15-3000 or about 320-31250. In some embodiments, the molar ratio is based on the amount of c-Met inhibitor:synthetic miRNA molecule provided in a single administration, a single day, a single week, 14 days, 21 days, or 28 days. In some embodiments, (a) the molar ratio of c-Met inhibitor:synthetic miRNA molecule, based on the amount of c-Met inhibitor and synthetic miRNA molecule provided to the subject in a single day, is about 15-764; or (b) the molar ratio of c-Met inhibitor:synthetic miRNA molecule, based on the amount of c-Met inhibitor and synthetic miRNA molecule provided to the subject over a single week, is about 22-2674. In some embodiments, the molar ratio of c-Met inhibitor:synthetic miRNA molecule, based on the amount of c-Met inhibitor and synthetic miRNA molecule provided to the subject in a single day, is about 15, 21, 27, 31, 36, 41, 51, 55, 62, 73, 77, 82, 93, 102, 110, 123, 127, 146, 154, 164, 204, 218, 255, 306, 309, 463, 509, or 764. In some embodiments, the molar ratio of c-Met inhibitor:synthetic miRNA molecule, based on the amount of c-Met inhibitor and synthetic miRNA molecule administered to the subject over a single week, is about 22, 29, 38, 43, 51, 54, 58, 71, 72, 77, 86, 96, 102, 108, 115, 127, 130, 143, 144, 153, 173, 178, 192, 204, 216, 230, 255, 270, 285, 288, 306, 324, 357, 383, 428, 431, 432, 446, 509, 540, 575, 648, 713, 764, 891, 1070, 1070, 1081, 1621, 1783, or 2674. In some embodiments, the c-Met inhibitor and synthetic miRNA molecule are synergistic. In some embodiments, the c-Met inhibitor and synthetic miRNA molecule have a combination index (CI)<1. In some embodiments, the combination index (CI) is less than about 0.80, 0.75, 0.70, 0.65, 0.60, 0.55, 0.50, 0.45, 0.40, 0.35, 0.30, 0.25, or 0.20. In some embodiments, the synthetic miRNA molecule is administered prior to the c-Met inhibitor. In some embodiments, the synthetic miRNA molecule is administered after the c-Met inhibitor. In some embodiments, the cancer has primary resistance to the c-Met inhibitor. In some embodiments, the cancer has secondary resistance to the c-Met inhibitor. In some embodiments, the methods further comprise identifying the cancer as having resistance to a c-Met inhibitor. In some embodiments, the c-Met inhibitor and/or the synthetic miRNA molecule are administered to a cancer cell in vivo or ex vivo. In some embodiments, the synthetic miRNA molecule is administered in a liposomal formulation. In some embodiments, the synthetic miRNA molecule is administered to the individual QDx3, QDx4, or QDx5. In some embodiments, the synthetic miRNA molecule is administered to the individual QDx3, QDx4, or QDx5 for 3 weeks. In some embodiments, the synthetic miRNA molecule is administered to the individual QDx5. In some embodiments, the synthetic miRNA molecule is administered to the individual QDx5 for 3 weeks. In some embodiments, the synthetic miRNA molecule is administered to the individual BIW. In some embodiments, the synthetic miRNA molecule is administered to the individual BIW for 3 weeks. In some embodiments, the methods further comprise administering to the individual dexamethasone.

Disclosed herein, in certain embodiments, are compositions comprising: (a) a c-Met inhibitor; and (b) a synthetic miRNA molecule, comprising: (i) an active strand comprising a sequence at least 80% identical to a mature miRNA; and (ii) a separate complementary strand that is at least 60% complementary to the active strand. In some embodiments, the passenger strand of the synthetic miRNA molecule comprises a 5′ terminal cap. In some embodiments, the 5′ terminal cap is a lower alkylamine. In some embodiments, the 5′ terminal cap is NH₂—(CH₂)₆—O—. In some embodiments, the mature miRNA molecule is miR-34a (SEQ ID NO: 1). In some embodiments, the mature miRNA molecule is miR-34b (SEQ ID NO: 2). In some embodiments, the mature miRNA molecule is miR-34c (SEQ ID NO: 3). In some embodiments, the mature miRNA molecule comprises a miR-34 consensus sequence (SEQ ID NO: 4). In some embodiments, the mature miRNA molecule is miR-449a (SEQ ID NO: 5). In some embodiments, the mature miRNA molecule is miR-449b (SEQ ID NO: 6). In some embodiments, the mature miRNA molecule is miR-449c (SEQ ID NO: 7). In some embodiments, the mature miRNA molecule comprises a miR-449 consensus sequence (SEQ ID NO: 8). In some embodiments, the mature miRNA molecule comprises a miR-34/449 seed sequence (SEQ ID NO: 9). In some embodiments, the c-Met inhibitor is selected from the group consisting of: ARQ197 (Tivantinib), GSK/1363089/XL880 (Foretinib), XL184 (Cabozantinib), HMPL-504/AZD6094/volitinib (Savolitinib), MSC2156119J (EMD 1214063, Tepotinib), LY2801653 (Merestinib), AMG 337, INCB28060 (Capmatinib), AMG 458, PF-04217903, PF-02341066 (Crizotinib), E7050 (Golvatinib), MK-2461, BMS-777607, JNJ-38877605, EMD1214063 (MSC2156119J; Tepotinib), SOMG-833, or pharmaceutically acceptable salts thereof. In some embodiments, the c-Met inhibitor is an ATP non-competitive c-Met inhibitor. In some embodiments, the c-Met inhibitor is ARQ197 (tivantinib). In some embodiments, the c-Met inhibitor is EMD1214063 (MSC2156119J; Tepotinib). In some embodiments, the composition further comprises a liposome. In some embodiments, the combination further comprises dexamethasone.

Disclosed herein, in certain embodiments, are combinations of: (a) a c-Met inhibitor; and (b) a synthetic miRNA molecule, comprising: (i) an active strand comprising a sequence at least 80% identical to a mature miRNA; and (ii) a separate complementary strand that is at least 60% complementary to the active strand, for use in treating a cancer. In some embodiments, the passenger strand of the synthetic miRNA molecule comprises a 5′ terminal cap. In some embodiments, the 5′ terminal cap is a lower alkylamine. In some embodiments, the 5′ terminal cap is NH₂—(CH₂)₆—O—. In some embodiments, the mature miRNA molecule is miR-34a (SEQ ID NO: 1). In some embodiments, the mature miRNA molecule is miR-34b (SEQ ID NO: 2). In some embodiments, the mature miRNA molecule is miR-34c (SEQ ID NO: 3). In some embodiments, the mature miRNA molecule comprises a miR-34 consensus sequence (SEQ ID NO: 4). In some embodiments, the mature miRNA molecule is miR-449a (SEQ ID NO: 5). In some embodiments, the mature miRNA molecule is miR-449b (SEQ ID NO: 6). In some embodiments, the mature miRNA molecule is miR-449c (SEQ ID NO: 7). In some embodiments, the mature miRNA molecule comprises a miR-449 consensus sequence (SEQ ID NO: 8). In some embodiments, the mature miRNA molecule comprises a miR-34/449 seed sequence (SEQ ID NO: 9). In some embodiments, the cancer is pancreatic, gastric, lung, thyroid, brain, kidney, head and neck, or liver cancer. In some embodiments, the cancer is liver cancer. In some embodiments, the liver cancer is primary liver cancer. In some embodiments, the liver cancer is hepatocellular carcinoma (HCC). In some embodiments, the c-Met inhibitor and the synthetic miRNA molecule are administered concurrently. In some embodiments, the c-Met inhibitor and the synthetic miRNA molecule are administered sequentially. In some embodiments, the c-Met inhibitor and the synthetic miRNA molecule are administered in a unified dosage form. In some embodiments, the c-Met inhibitor and the synthetic miRNA molecule are administered in separate dosage forms. In some embodiments, the c-Met inhibitor is selected from the group consisting of: ARQ197 (Tivantinib), GSK/1363089/XL880 (Foretinib), XL184 (Cabozantinib), HMPL-504/AZD6094/volitinib (Savolitinib), MSC2156119J (EMD 1214063, Tepotinib), LY2801653 (Merestinib), AMG 337, INCB28060 (Capmatinib), AMG 458, PF-04217903, PF-02341066 (Crizotinib), E7050 (Golvatinib), MK-2461, BMS-777607, JNJ-38877605, EMD1214063 (MSC2156119J; Tepotinib), SOMG-833, or pharmaceutically acceptable salts thereof. In some embodiments, the c-Met inhibitor is an ATP non-competitive c-Met inhibitor. In some embodiments, the c-Met inhibitor is ARQ197 (tivantinib). In some embodiments, the c-Met inhibitor is EMD1214063 (MSC2156119J; Tepotinib). In some embodiments, the c-Met inhibitor and synthetic miRNA molecule are administered to an individual in need thereof in molar ratio of about 15-3000 or about 320-31250. In some embodiments, the molar ratio is based on the amount of c-Met inhibitor:synthetic miRNA molecule provided in a single administration, a single day, a single week, 14 days, 21 days, or 28 days. In some embodiments, (a) the molar ratio of c-Met inhibitor:synthetic miRNA molecule, based on the amount of c-Met inhibitor and synthetic miRNA molecule provided to the subject in a single day, is about 15-764; or (b) the molar ratio of c-Met inhibitor:synthetic miRNA molecule, based on the amount of c-Met inhibitor and synthetic miRNA molecule provided to the subject over a single week, is about 22-2674. In some embodiments, the molar ratio of c-Met inhibitor:synthetic miRNA molecule, based on the amount of c-Met inhibitor and synthetic miRNA molecule provided to the subject in a single day, is about 15, 21, 27, 31, 36, 41, 51, 55, 62, 73, 77, 82, 93, 102, 110, 123, 127, 146, 154, 164, 204, 218, 255, 306, 309, 463, 509, or 764. In some embodiments, the molar ratio of c-Met inhibitor:synthetic miRNA molecule, based on the amount of c-Met inhibitor and synthetic miRNA molecule administered to the subject over a single week, is about 22, 29, 38, 43, 51, 54, 58, 71, 72, 77, 86, 96, 102, 108, 115, 127, 130, 143, 144, 153, 173, 178, 192, 204, 216, 230, 255, 270, 285, 288, 306, 324, 357, 383, 428, 431, 432, 446, 509, 540, 575, 648, 713, 764, 891, 1070, 1070, 1081, 1621, 1783, or 2674. In some embodiments, the c-Met inhibitor and synthetic miRNA molecule are synergistic. In some embodiments, the c-Met inhibitor and synthetic miRNA molecule have a combination index (CI)<1. In some embodiments, the combination index (CI) is less than about 0.80, 0.75, 0.70, 0.65, 0.60, 0.55, 0.50, 0.45, 0.40, 0.35, 0.30, 0.25, or 0.20. In some embodiments, the synthetic miRNA molecule is administered prior to the c-Met inhibitor. In some embodiments, the synthetic miRNA molecule is administered after the c-Met inhibitor. In some embodiments, the cancer has primary resistance to the c-Met inhibitor. In some embodiments, the cancer has secondary resistance to the c-Met inhibitor. In some embodiments, the c-Met inhibitor and/or the synthetic miRNA molecule are administered to a cancer cell in vivo or ex vivo. In some embodiments, the synthetic miRNA molecule is administered in a liposomal formulation. In some embodiments, the synthetic miRNA molecule is administered to the individual QDx3, QDx4, or QDx5. In some embodiments, the synthetic miRNA molecule is administered to the individual QDx3, QDx4, or QDx5 for 3 weeks. In some embodiments, the synthetic miRNA molecule is administered to the individual QDx5. In some embodiments, the synthetic miRNA molecule is administered to the individual QDx5 for 3 weeks. In some embodiments, the synthetic miRNA molecule is administered to the individual BIW. In some embodiments, the synthetic miRNA molecule is administered to the individual BIW for 3 weeks. In some embodiments, the methods further comprise administering to the individual dexamethasone.

Disclosed herein, in certain embodiments, are methods of reducing, inhibiting or preventing cancer cell proliferation in an individual in need thereof, comprising: administering to the individual: (a) a c-Met inhibitor; and (b) a synthetic miRNA molecule, comprising: (i) an active strand comprising a sequence at least 80% identical to a mature miRNA; and (ii) a separate complementary strand that is at least 60% complementary to the active strand. In some embodiments, the passenger strand of the synthetic miRNA molecule comprises a 5′ terminal cap. In some embodiments, the 5′ terminal cap is a lower alkylamine. In some embodiments, the 5′ terminal cap is NH₂—(CH₂)₆—O—. In some embodiments, the mature miRNA molecule is miR-34a (SEQ ID NO: 1). In some embodiments, the mature miRNA molecule is miR-34b (SEQ ID NO: 2). In some embodiments, the mature miRNA molecule is miR-34c (SEQ ID NO: 3). In some embodiments, the mature miRNA molecule comprises a miR-34 consensus sequence (SEQ ID NO: 4). In some embodiments, the mature miRNA molecule is miR-449a (SEQ ID NO: 5). In some embodiments, the mature miRNA molecule is miR-449b (SEQ ID NO: 6). In some embodiments, the mature miRNA molecule is miR-449c (SEQ ID NO: 7). In some embodiments, the mature miRNA molecule comprises a miR-449 consensus sequence (SEQ ID NO: 8). In some embodiments, the mature miRNA molecule comprises a miR-34/449 seed sequence (SEQ ID NO: 9). In some embodiments, the cancer cell is a pancreatic cancer cell, gastric cancer cell, lung cancer cell, thyroid cancer cell, brain cancer cell, kidney cancer cell, head and neck cancer cell, or liver cancer cell. In some embodiments, the cancer cell is a liver cancer cell. In some embodiments, the cancer cell is a hepatocellular carcinoma (HCC) cell. In some embodiments, the c-Met inhibitor and the synthetic miRNA molecule are administered concurrently. In some embodiments, the c-Met inhibitor and the synthetic miRNA molecule are administered sequentially. In some embodiments, the c-Met inhibitor and the synthetic miRNA molecule are administered in a unified dosage form. In some embodiments, the c-Met inhibitor and the synthetic miRNA molecule are administered in separate dosage forms. In some embodiments, the c-Met inhibitor is selected from the group consisting of: ARQ197 (Tivantinib), GSK/1363089/XL880 (Foretinib), XL184 (Cabozantinib), HMPL-504/AZD6094/volitinib (Savolitinib), MSC2156119J (EMD 1214063, Tepotinib), LY2801653 (Merestinib), AMG 337, INCB28060 (Capmatinib), AMG 458, PF-04217903, PF-02341066 (Crizotinib), E7050 (Golvatinib), MK-2461, BMS-777607, JNJ-38877605, EMD1214063 (MSC2156119J; Tepotinib), SOMG-833, or pharmaceutically acceptable salts thereof. In some embodiments, the c-Met inhibitor is an ATP non-competitive c-Met inhibitor. In some embodiments, the c-Met inhibitor is ARQ197 (tivantinib). In some embodiments, the c-Met inhibitor is EMD1214063 (MSC2156119J; Tepotinib). In some embodiments, the c-Met inhibitor and synthetic miRNA molecule are administered in molar ratio of about 15-3000 or about 320-31250. In some embodiments, the molar ratio is based on the amount of c-Met inhibitor:synthetic miRNA molecule provided in a single administration, a single day, a single week, 14 days, 21 days, or 28 days. In some embodiments, (a) the molar ratio of c-Met inhibitor:synthetic miRNA molecule, based on the amount of c-Met inhibitor and synthetic miRNA molecule provided to the subject in a single day, is about 15-764; or (b) the molar ratio of c-Met inhibitor:synthetic miRNA molecule, based on the amount of c-Met inhibitor and synthetic miRNA molecule provided to the subject over a single week, is about 22-2674. In some embodiments, the molar ratio of c-Met inhibitor:synthetic miRNA molecule, based on the amount of c-Met inhibitor and synthetic miRNA molecule provided to the subject in a single day, is about 15, 21, 27, 31, 36, 41, 51, 55, 62, 73, 77, 82, 93, 102, 110, 123, 127, 146, 154, 164, 204, 218, 255, 306, 309, 463, 509, or 764. In some embodiments, the molar ratio of c-Met inhibitor:synthetic miRNA molecule, based on the amount of c-Met inhibitor and synthetic miRNA molecule administered to the subject over a single week, is about 22, 29, 38, 43, 51, 54, 58, 71, 72, 77, 86, 96, 102, 108, 115, 127, 130, 143, 144, 153, 173, 178, 192, 204, 216, 230, 255, 270, 285, 288, 306, 324, 357, 383, 428, 431, 432, 446, 509, 540, 575, 648, 713, 764, 891, 1070, 1070, 1081, 1621, 1783, or 2674. In some embodiments, the c-Met inhibitor and synthetic miRNA molecule are synergistic. In some embodiments, the c-Met inhibitor and synthetic miRNA molecule have a combination index (CI)<1. In some embodiments, the combination index (CI) is less than about 0.80, 0.75, 0.70, 0.65, 0.60, 0.55, 0.50, 0.45, 0.40, 0.35, 0.30, 0.25, or 0.20. In some embodiments, the synthetic miRNA molecule is administered prior to the c-Met inhibitor. In some embodiments, the synthetic miRNA molecule is administered after the c-Met inhibitor. In some embodiments, the cancer has primary resistance to the c-Met inhibitor. In some embodiments, the cancer has secondary resistance to the c-Met inhibitor. In some embodiments, the cancer has secondary resistance to the c-Met inhibitor. In some embodiments, the c-Met inhibitor and/or the synthetic miRNA molecule are administered to a cancer cell in vivo or ex vivo. In some embodiments, the synthetic miRNA molecule is administered in a liposomal formulation. In some embodiments, the synthetic miRNA molecule is administered to the individual QDx3, QDx4, or QDx5. In some embodiments, the synthetic miRNA molecule is administered to the individual QDx3, QDx4, or QDx5 for 3 weeks. In some embodiments, the synthetic miRNA molecule is administered to the individual QDx5. In some embodiments, the synthetic miRNA molecule is administered to the individual QDx5 for 3 weeks. In some embodiments, the synthetic miRNA molecule is administered to the individual BIW. In some embodiments, the synthetic miRNA molecule is administered to the individual BIW for 3 weeks. In some embodiments, the methods further comprise administering to the individual dexamethasone.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which principles of the invention are utilized, and the accompanying drawings of which:

FIGS. 1A-C provide graphs that exemplify dose response curves of tivantinib alone (FIG. 1A) and a miR-34 mimic alone (FIG. 1B) in Hep3B, C3A, HepG2, Huh7 HCC, and EMD1214063 alone (FIG. 1C) in C3A and SK-Hep1 HCC cells. Cells were treated with tivantinib alone at indicated concentrations, and cell proliferation was measured 3 days post drug treatment. Non-linear dose-response curves and IC50 values were calculated using GraphPad. (variable slope)

FIGS. 2A-D provide graphs that illustrate tivantinib and a miR-34a mimic synergizing in Hep3B HCC cells. FIG. 2A illustrates combination index (CI) analysis. CI values were generated by non-linear regression methods. Trendlines indicate CI values at any given effect (Fa, fraction affected, % inhibition), and symbols represent CI values derived from actual data points. CI=1, additivity; CI>1, antagonism; CI<1, synergy. FIG. 2B illustrates curve shift analysis. Data derived from non-linear dose-response curves were normalized to IC50 values of the single agents (IC₅₀ eq) and plotted in the same graph. Left and right shifts of the dose-response curves of the combination relative to the dose-response curves of the single agents indicate synergy or antagonism, respectively. FIGS. 2C and 2D illustrate isobologram analysis. The diagonal, dotted line indicates additivity, and the square symbol shows dose requirements to achieve 50% and 80% cancer cell inhibition, respectively. Data points below the line of additivity indicate synergy, data points above denote antagonism. Each data point is an average of raw data in triplicates, and the experiment has been repeated three times in each cell line.

FIGS. 3A-D illustrates tivantinib and a miR-34a mimic synergizing in HepG2 HCC cells. FIG. 3A illustrates combination index (CI) analysis. FIG. 3B illustrates curve shift analysis. FIGS. 3C and 3D illustrate isobologram analysis at dose requirements to achieve 50% and 80% cancer cell inhibition, respectively. The generation and analysis of this information is the same as described in connection with FIGS. 2A-D above.

FIGS. 4A-D illustrates tivantinib and a miR-34a mimic synergizing in C3A HCC cells. FIG. 4A illustrates combination index (CI) analysis. FIG. 4B illustrates curve shift analysis. FIGS. 4C and 4D illustrate isobologram analysis at dose requirements to achieve 50% and 80% cancer cell inhibition, respectively. The generation and analysis of this information is the same as described in connection with FIGS. 2A-D above.

FIGS. 5A-D illustrates tivantinib and a miR-34a mimic synergizing in Huh7 HCC cells. FIG. 5A illustrates combination index (CI) analysis. FIG. 5B illustrates curve shift analysis. FIGS. 5C and 5D illustrate isobologram analysis at dose requirements to achieve 50% and 80% cancer cell inhibition, respectively. The generation and analysis of this information is the same as described in connection with FIGS. 2A-D above.

FIG. 6A-D illustrates synergistic effects between tivantinib and a miR-34a mimic at multiple ratios in Hep3B cells. FIG. 6A illustrates combination index plot of various drug ratios. FIG. 6B illustrates curve shift analysis of various drug ratios. FIG. 6C illustrates isobologram at 50% and FIG. 6D illustrates isobologram at 80% cancer cell inhibition.

FIG. 7A-D illustrates synergistic effects between tivantinib and a miR-34a mimic at multiple ratios in HepG2 cells. FIG. 7A illustrates combination index plot of various tivantinib and miR-34a mimic drug ratios. FIG. 7B illustrates curve shift analysis of various drug ratios. FIG. 7C illustrates isobologram at 50% and FIG. 7D illustrates isobologram at 80% cancer cell inhibition.

FIG. 8A-C illustrates EMD1214063 and miR-Rx34 synergizing in C3A HCC cells. FIG. 8A illustrates combination index (CI) analysis. FIG. 8B illustrates curve shift analysis. FIG. 8C illustrates isobologram analysis at dose requirements to achieve 50% cancer cell inhibition. The generation and analysis of this information is the same as described in connection with FIGS. 2A-C above.

FIG. 9A-C illustrates EMD1214063 and miR-Rx34 synergizing in SK-Hep1 HCC cells. FIG. 9A illustrates combination index (CI) analysis. FIG. 9B illustrates curve shift analysis. FIG. 9C illustrates isobologram analysis at dose requirements to achieve 50% cancer cell inhibition. The generation and analysis of this information is the same as described in connection with FIGS. 2A-D above.

FIG. 10A-C illustrates synergistic effects between EMD1214063 and miR-Rx34 at multiple ratios in C3A HCC cells. FIG. 10A illustrates combination index plot of various drug ratios. FIG. 10B illustrates curve shift analysis of various drug ratios. FIG. 10C illustrates isobologram at 50% cancer cell inhibition.

FIG. 11A-C illustrates synergistic effects between EMD1214063 and miR-Rx34 at multiple ratios in SK-Hep1 HCC cells. FIG. 11A illustrates combination index plot of various drug ratios. FIG. 11B illustrates curve shift analysis of various drug ratios. FIG. 11C illustrates isobologram at 50% cancer cell inhibition.

### DETAILED DESCRIPTION OF THE INVENTION

Disclosed herein, in some embodiments, are combinations of c-Met inhibitor and a synthetic miRNA molecule. In some embodiments, the synthetic miRNA molecule comprises (a) an active strand comprising a sequence at least 80% identical to a mature miRNA; and (b) a separate complementary strand that is at least 60% complementary to the active strand. In some embodiments, the mature miRNA is miR-34a (SEQ ID NO: 1). In some embodiments, the mature miRNA is miR-34b (SEQ ID NO: 2). In some embodiments, the mature miRNA is miR-34c (SEQ ID NO: 3). In some embodiments, the mature miRNA comprises a miR-34 consensus sequence (SEQ ID NO: 4). In some embodiments, the mature miRNA is miR-449. In some embodiments, the mature miRNA is miR-449a (SEQ ID NO: 5). In some embodiments, the mature miRNA is miR-449b (SEQ ID NO: 6). In some embodiments, the mature miRNA is miR-449c (SEQ ID NO: 7). In some embodiments, the mature miRNA comprises a miR-449 consensus sequence (SEQ ID NO: 8). In some embodiments, the mature miRNA comprises a miR-34/449 seed sequence (SEQ ID NO: 9).

Disclosed herein are methods of treating a cancer in an individual in need thereof. In some embodiments, the methods comprise administering to the individual a c-Met inhibitor and a synthetic miRNA molecule. In some embodiments, the synthetic miRNA molecule comprises (a) an active strand comprising a sequence at least 80% identical to a mature miRNA; and (b) a separate complementary strand that is at least 60% complementary to the active strand. In some embodiments, the mature miRNA is miR-34a (SEQ ID NO: 1). In some embodiments, the mature miRNA is miR-34b (SEQ ID NO: 2). In some embodiments, the mature miRNA is miR-34c (SEQ ID NO: 3). In some embodiments, the mature miRNA comprises a miR-34 consensus sequence (SEQ ID NO: 4). In some embodiments, the mature miRNA is miR-449. In some embodiments, the mature miRNA is miR-449a (SEQ ID NO: 5). In some embodiments, the mature miRNA is miR-449b (SEQ ID NO: 6). In some embodiments, the mature miRNA is miR-449c (SEQ ID NO: 7). In some embodiments, the mature miRNA comprises a miR-449 consensus sequence (SEQ ID NO: 8). In some embodiments, the mature miRNA comprises a miR-34/449 seed sequence (SEQ ID NO: 9). In some embodiments, the cancer is pancreatic, gastric, lung, thyroid, brain, kidney, head and neck, or liver cancer. In some embodiments, the cancer is liver cancer. In some embodiments, the cancer is primary liver cancer. In some embodiments, the cancer is HCC.

### DEFINITIONS

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “subject,” “individual,” and “patient” are used interchangeably herein to refer to a vertebrate, for example, a mammal. Mammals include, but are not limited to, murines, simians, humans, farm animals, sport animals, and pets. Tissues, cells, and their progeny of a biological entity obtained in vivo or cultured in vitro are also encompassed. Designation as a “subject” does not necessarily entail supervision of a medical professional.

A “synergistic” or “synergizing” effect can be such that the one or more effects of the combination compositions are greater than the one or more effects of each component alone, or they can be greater than the sum of the one or more effects of each component alone. In some embodiments, the synergistic effect is about, or greater than about 10%, 20%, 30%, 50%, 75%, 100%, 110%, 120%, 150%, 200%, 250%, 350%, or 500% or even more than the effect on an individual with one of the components alone, or the additive effects of each of the components when administered individually. The effect can be any of the measurable effects described herein.

Synthetic miRNAs Molecule

Disclosed herein are combinations of c-Met inhibitors and a synthetic microRNA molecule comprising (a) an active strand comprising a sequence at least 80% identical to a mature miRNA; and (b) a separate complementary strand that is at least 60% complementary to the active strand. In some embodiments, the c-Met inhibitor is ARQ197 (Tivantinib). In some embodiments, the c-Met inhibitor is EMD1214063 (MSC2156119J; Tepotinib). In some embodiments, the active strand comprises a sequence at least 80% identical to miR-34a.

Further disclosed herein, are methods of treating a cancer in an individual in need thereof comprising administering to the individual a c-Met inhibitor and a synthetic miRNA molecule comprising (a) an active strand comprising a sequence at least 80% identical to a mature miRNA; and (b) a separate complementary strand that is at least 60% complementary to the active strand. In some embodiments, the c-Met inhibitor is ARQ197 (Tivantinib). In some embodiments, the c-Met inhibitor is EMD1214063 (MSC2156119J; Tepotinib). In some embodiments, the active strand comprises a sequence at least 80% identical to miR-34a. In some embodiments, the cancer is selected from the group consisting of: pancreatic, gastric, lung, thyroid, brain, kidney, head and neck, and liver cancer.

MicroRNAs (miRNAs) are small non-coding, naturally occurring RNA molecules that post-transcriptionally modulate gene expression and determine cell fate by regulating multiple gene products and cellular pathways. miRNAs interfere with gene expression by degrading the mRNA transcript by blocking the protein translation machinery. miRNAs target mRNAs with sequences that are fully or partially complementary which endows these regulatory RNAs with the ability to target a broad but nevertheless specific set of mRNAs. To date, there are 1,500 human annotated miRNA genes with roles in processes as diverse as cell proliferation, differentiation, apoptosis, stem cell development, and immune function. Often, the misregulation of miRNAs can contribute to the development of human diseases including cancer. miRNAs deregulated in cancer can function as bona fide tumor suppressors or oncogenes. A single miRNA can target multiple oncogenes and oncogenic signaling pathways, and translating this ability into a future therapeutic may hold the promise of creating a remedy that is effective against tumor heterogeneity. Thus, miRNAs have the potential of becoming powerful therapeutic agents for cancer that act in accordance with our current understanding of cancer as a “pathway disease” that can only be successfully treated when intervening with multiple cancer pathways.

In some embodiments, a synthetic miRNA molecule is a microRNA mimic. In some embodiments, the synthetic miRNA molecule is administered by injection or transfusion. In some embodiments, the synthetic miRNA molecule is provided in a vector (e.g., using a gene therapy methodology). Representative synthetic miRNA molecule sequences are provided in Table 1 below.

TABLE 1
microRNA Sequences and Sequence Identification
Numbers

| microRNA | Sequence | SEQ ID NO: |
|---|---|---|
| miR-34a | UGGCAGUGUCUUAGCUGGUUGUU | SEQ ID NO: 1 |
| miR-34b | UAGGCAGUGUCAUUAGCUGAUUG | SEQ ID NO: 2 |
| miR-34c | AGGCAGUGUAGUUAGCUGAUUGC | SEQ ID NO: 3 |
| miR-34 | *GGCAGUGU*UUAGCUG*-UUG* | SEQ ID NO: 4 |
| consensus | | |
| miR-449a | UGGCAGUGUAUUGUUAGCUGGU | SEQ ID NO: 5 |
| miR-449b | AGGCAGUGUAUUGUUAGCUGGC | SEQ ID NO: 6 |
| miR-449c | UAGGCAGUGUAUUGCUAGCGGC | SEQ ID NO: 7 |
| | UGU | |
| miR-449 | UGGCAGUGUAUUG*UAGC*-G*G | SEQ ID NO: 8 |
| consensus | | |
| miR-34/449 | GGCAGUG | SEQ ID NO: 9 |
| seed | | |

″*″ denotes a deletion of any nucleotide(s). miR-34/449 seed sequences are shown in bold highlighting.

In some embodiments, the synthetic miRNA molecule is 7-130 nucleotides long, double stranded RNA molecules. In some embodiments, a synthetic miRNA molecule can be 7, 8, 9, 10, 12, 13, 14, 15, 16, 17, 18, 19, 20, 21, 22, 23, 24, 25, 26, 27, 28, 29, 30, 7-30, 7-25, 15-30, 15-25, 17-30, or 17-25 nucleotides long.

In some embodiments, the synthetic miRNA molecule is two separate strands (i.e., an active strand and a separate passenger strand). In some embodiments, the synthetic miRNA molecule is a hairpin structure.

In some embodiment, the active strand comprises or consists of a sequence which is identical or substantially identical to a mature microRNA sequence. In some embodiments, “substantially identical”, as used herein, means that the sequence is at least 80% identical to the mature microRNA sequence. In some embodiments, the mature microRNA sequence is miR-34a (SEQ ID NO: 1). In some embodiments, the mature microRNA sequence is miR-34b (SEQ ID NO: 2). In some embodiments, the mature microRNA sequence is miR-34c (SEQ ID NO: 3). In some embodiments, the mature microRNA sequence is miR-449a (SEQ ID NO: 5). In some embodiments, the mature microRNA sequence is miR-449b (SEQ ID NO: 6). In some embodiments, the mature microRNA sequence is miR-449c (SEQ ID NO: 7).

In some embodiments, the active strand comprises or consists of a sequence that is at least 80% identical to miR-34a (SEQ ID NO: 1). In some embodiments, the active strand comprises or consists of a sequence that is at least 80% identical to miR-34b (SEQ ID NO: 2). In some embodiments, the active strand comprises or consists of a sequence that is at least 80% identical to miR-34c (SEQ ID NO: 3). In some embodiments, the active strand comprises or consists of a sequence that is at least 80% identical to miR-449a (SEQ ID NO: 5). In some embodiments, the active strand comprises or consists of a sequence that is at least 80% identical to miR-449b (SEQ ID NO: 6). In some embodiments, the active strand comprises or consists of a sequence that is at least 80% identical to miR-449c (SEQ ID NO: 7). In some embodiments, the active strand comprises or consists of a sequence which is identical or substantially identical to the miR-34/449 seed sequence (SEQ ID NO: 9). In some embodiments, the active strand comprises or consists of a sequence which is identical or substantially identical to the miR-34 consensus sequence (SEQ ID NO: 4). In some embodiments, the active strand comprises or consists of a sequence which is identical or substantially identical to the miR-34 consensus sequence (SEQ ID NO: 8).

In some embodiments, the active strand comprises or consists of a sequence that is at least 85% identical to miR-34a (SEQ ID NO: 1). In some embodiments, the active strand comprises or consists of a sequence that is at least 85% identical to miR-34b (SEQ ID NO: 2). In some embodiments, the active strand comprises or consists of a sequence that is at least 85% identical to miR-34c (SEQ ID NO: 3). In some embodiments, the active strand comprises or consists of a sequence that is at least 85% identical to miR-449a (SEQ ID NO: 5). In some embodiments, the active strand comprises or consists of a sequence that is at least 85% identical to miR-449b (SEQ ID NO: 6). In some embodiments, the active strand comprises or consists of a sequence that is at least 85% identical to miR-449c (SEQ ID NO: 7). In some embodiments, the active strand comprises or consists of a sequence which is identical or substantially identical to the miR-34/449 seed sequence (SEQ ID NO: 9). In some embodiments, the active strand comprises or consists of a sequence which is identical or substantially identical to the miR-34 consensus sequence (SEQ ID NO: 4). In some embodiments, the active strand comprises or consists of a sequence which is identical or substantially identical to the miR-34 consensus sequence (SEQ ID NO: 8).

In some embodiments, the active strand comprises or consists of a sequence that is at least 90% identical to miR-34a (SEQ ID NO: 1). In some embodiments, the active strand comprises or consists of a sequence that is at least 90% identical to miR-34b (SEQ ID NO: 2). In some embodiments, the active strand comprises or consists of a sequence that is at least 90% identical to miR-34c (SEQ ID NO: 3). In some embodiments, the active strand comprises or consists of a sequence that is at least 90% identical to miR-449a (SEQ ID NO: 5). In some embodiments, the active strand comprises or consists of a sequence that is at least 90% identical to miR-449b (SEQ ID NO: 6). In some embodiments, the active strand comprises or consists of a sequence that is at least 90% identical to miR-449c (SEQ ID NO: 7). In some embodiments, the active strand comprises or consists of a sequence which is identical or substantially identical to the miR-34/449 seed sequence (SEQ ID NO: 9). In some embodiments, the active strand comprises or consists of a sequence which is identical or substantially identical to the miR-34 consensus sequence (SEQ ID NO: 4). In some embodiments, the active strand comprises or consists of a sequence which is identical or substantially identical to the miR-34 consensus sequence (SEQ ID NO: 8).

In some embodiments, the active strand comprises or consists of a sequence that is at least 95% identical to miR-34a (SEQ ID NO: 1). In some embodiments, the active strand comprises or consists of a sequence that is at least 95% identical to miR-34b (SEQ ID NO: 2). In some embodiments, the active strand comprises or consists of a sequence that is at least 95% identical to miR-34c (SEQ ID NO: 3). In some embodiments, the active strand comprises or consists of a sequence that is at least 95% identical to miR-449a (SEQ ID NO: 5). In some embodiments, the active strand comprises or consists of a sequence that is at least 95% identical to miR-449b (SEQ ID NO: 6). In some embodiments, the active strand comprises or consists of a sequence that is at least 95% identical to miR-449c (SEQ ID NO: 7). In some embodiments, the active strand comprises or consists of a sequence which is identical or substantially identical to the miR-34/449 seed sequence (SEQ ID NO: 9). In some embodiments, the active strand comprises or consists of a sequence which is identical or substantially identical to the miR-34 consensus sequence (SEQ ID NO: 4). In some embodiments, the active strand comprises or consists of a sequence which is identical or substantially identical to the miR-34 consensus sequence (SEQ ID NO: 8).

In some embodiments, the active strand comprises or consists of a sequence that is identical to miR-34a (SEQ ID NO: 1). In some embodiments, the active strand comprises or consists of a sequence that is identical to miR-34b (SEQ ID NO: 2). In some embodiments, the active strand comprises or consists of a sequence that is identical to miR-34c (SEQ ID NO: 3). In some embodiments, the active strand comprises or consists of a sequence that is identical to miR-449a (SEQ ID NO: 5). In some embodiments, the active strand comprises or consists of a sequence that is identical to miR-449b (SEQ ID NO: 6). In some embodiments, the active strand comprises or consists of a sequence that is identical to miR-449c (SEQ ID NO: 7). In some embodiments, the active strand comprises or consists of a sequence which is identical or substantially identical to the miR-34/449 seed sequence (SEQ ID NO: 9). In some embodiments, the active strand comprises or consists of a sequence which is identical or substantially identical to the miR-34 consensus sequence (SEQ ID NO: 4). In some embodiments, the active strand comprises or consists of a sequence which is identical or substantially identical to the miR-34 consensus sequence (SEQ ID NO: 8).

In some embodiments, the passenger strand comprises a sequence that is at least 60% complementary to the active strand. In some embodiments, the passenger strand comprises a sequence that is at least 65% complementary to the active strand. In some embodiments, the passenger strand comprises a sequence that is at least 70% complementary to the active strand. In some embodiments, the passenger strand comprises a sequence that is at least 75% complementary to the active strand. In some embodiments, the passenger strand comprises a sequence that is at least 80% complementary to the active strand. In some embodiments, the passenger strand comprises a sequence that is at least 85% complementary to the active strand. In some embodiments, the passenger strand comprises a sequence that is at least 90% complementary to the active strand. In some embodiments, the passenger strand comprises a sequence that is at least 95% complementary to the active strand. In some embodiments, the passenger strand comprises a sequence that is complementary to the active strand.

In some embodiments, the synthetic microRNA molecule is chemically modified or designed to comprise one or more specific sequence variations. In some embodiments, synthetic miRNA molecule has a 5′ terminal cap on the passenger strand. Any suitable cap may be used with the molecules disclosed herein. In some embodiments, the synthetic microRNA molecule comprises a lower alkylamine cap on the 5′ terminus of the passenger strand. In some embodiments, the synthetic microRNA molecule comprises aNH₂—(CH₂)₆—O— cap on the 5′ terminus of the passenger strand. In some embodiments, the synthetic microRNA molecule comprises a mismatch at the first and/or second nucleotide of the passenger strand. In some embodiments, at least one nucleotide of the passenger strand comprises a sugar modification. In some embodiments, at least one nucleotide of the active strand comprises a sugar modification. In some embodiments, at least one nucleotide of the passenger strand and at least one nucleotide if the active strand comprises a sugar modification. Additional non-limiting examples of chemical modifications include backbone modifications (e.g., phosphorothioate, morpholinos), ribose modifications (e.g., 2′-OMe, 2′-Me, 2′-F, 2′-4′-locked/bridged sugars (e.g., LNA, ENA, UNA), and nucleobase modifications.

In some embodiments, the synthetic miRNA molecule is between 17 and 30 nucleotides in length and comprises (i) an active strand comprising or consisting of a sequence from 5′ to 3′ that is at least 80% identical to SEQ ID NO: 1, and (ii) a separate passenger strand comprising a sequence from 5′ to 3′ that is at least 60% complementary to the active strand. In some embodiments, the passenger strand comprises a 5′ terminal cap. In some embodiments, the 5′ terminal cap is a lower alkylamine.

In some embodiments, the synthetic miRNA molecule is between 17 and 30 nucleotides in length and comprises (i) an active strand comprising or consisting of a sequence from 5′ to 3′ that is at least 80% identical to SEQ ID NO: 2, and (ii) a separate passenger strand comprising or consisting of a sequence from 5′ to 3′ that is at least 60% complementary to the active strand. In some embodiments, the passenger strand comprises a 5′ terminal cap. In some embodiments, the 5′ terminal cap is a lower alkylamine.

In some embodiments, the synthetic miRNA molecule is between 17 and 30 nucleotides in length and comprises (i) an active strand comprising or consisting of a sequence from 5′ to 3′ that is at least 80% identical to SEQ ID NO: 3, and (ii) a separate passenger strand comprising or consisting of a sequence from 5′ to 3′ that is at least 60% complementary to the active strand. In some embodiments, the passenger strand comprises a 5′ terminal cap. In some embodiments, the 5′ terminal cap is a lower alkylamine.

In some embodiments, the synthetic miRNA molecule is between 17 and 30 nucleotides in length and comprises (i) an active strand comprising or consisting of a sequence from 5′ to 3′ that is at least 80% identical to SEQ ID NO: 4, and (ii) a separate passenger strand comprising or consisting of a sequence from 5′ to 3′ that is at least 60% complementary to the active strand. In some embodiments, the passenger strand comprises a 5′ terminal cap. In some embodiments, the 5′ terminal cap is a lower alkylamine.

In some embodiments, the synthetic miRNA molecule is between 17 and 30 nucleotides in length and comprises (i) an active strand comprising or consisting of a sequence from 5′ to 3′ that is at least 80% identical to SEQ ID NO: 5, and (ii) a separate passenger strand comprising or consisting of a sequence from 5′ to 3′ that is at least 60% complementary to the active strand. In some embodiments, the passenger strand comprises a 5′ terminal cap. In some embodiments, the 5′ terminal cap is a lower alkylamine.

In some embodiments, the synthetic miRNA molecule is between 17 and 30 nucleotides in length and comprises (i) an active strand comprising or consisting of a sequence from 5′ to 3′ that is at least 80% identical to SEQ ID NO: 6, and (ii) a separate passenger strand comprising or consisting of a sequence from 5′ to 3′ that is at least 60% complementary to the active strand. In some embodiments, the passenger strand comprises a 5′ terminal cap. In some embodiments, the 5′ terminal cap is a lower alkylamine.

In some embodiments, the synthetic miRNA molecule is between 17 and 30 nucleotides in length and comprises (i) an active strand comprising or consisting of a sequence from 5′ to 3′ that is at least 80% identical to SEQ ID NO: 7, and (ii) a separate passenger strand comprising or consisting of a sequence from 5′ to 3′ that is at least 60% complementary to the active strand. In some embodiments, the passenger strand comprises a 5′ terminal cap. In some embodiments, the 5′ terminal cap is a lower alkylamine.

In some embodiments, the synthetic miRNA molecule is between 17 and 30 nucleotides in length and comprises (i) an active strand comprising or consisting of a sequence from 5′ to 3′ that is at least 80% identical to SEQ ID NO: 8, and (ii) a separate passenger strand comprising or consisting of a sequence from 5′ to 3′ that is at least 60% complementary to the active strand. In some embodiments, the passenger strand comprises a 5′ terminal cap. In some embodiments, the 5′ terminal cap is a lower alkylamine.

In some embodiments, the synthetic miRNA molecule is between 17 and 30 nucleotides in length and comprises (i) an active strand comprising or consisting of a sequence from 5′ to 3′ that is at least 80% identical to SEQ ID NO: 9, and (ii) a separate passenger strand comprising or consisting of a sequence from 5′ to 3′ that is at least 60% complementary to the active strand. In some embodiments, the passenger strand comprises a 5′ terminal cap. In some embodiments, the 5′ terminal cap is a lower alkylamine.

In some embodiments, the synthetic miRNA molecule comprises a sequence that is at least 80% identical to at least one of SEQ ID NO:1-9. In some embodiments, the synthetic miRNA molecule comprises a sequence that is at least 85% identical to at least one of SEQ ID NO:1-9. In some embodiments, the synthetic miRNA molecule comprises a sequence that is at least 90% identical to at least one of SEQ ID NO:1-9. In some embodiments, the synthetic miRNA molecule comprises a sequence that is at least 95% identical to at least one of SEQ ID NO:1-9. In some embodiments, the synthetic miRNA molecule comprises a sequence that is at least 100% identical to at least one of SEQ ID NO:1-9. In some embodiments, the synthetic miRNA molecule comprises a sequence that differs from at least one of SEQ ID NO:1-9 by 1, 2, 3, 4, 5, 6, 7, 8, 9, or 10 nucleobases.

In some embodiments, the synthetic miRNA molecule comprises a single polynucleotide or a double stranded polynucleotide. In some embodiments, the synthetic miRNA molecule comprises a hairpin polynucleotide.

In some embodiments, the synthetic miRNA molecule is between 17 and 30 nucleotides in length and comprises one or more of the following (i) a 5′ terminal cap on the passenger strand; (ii) one or more sugar modifications in the first or last 1 to 6 residues of the passenger strand; or (iii) non-complementarity between one or more nucleotides in the last 1 to 5 residues at the 3′ end of the passenger strand and the corresponding nucleotides of the active strand.

In some embodiments, the synthetic miRNA molecule is between 17 and 30 nucleotides in length and comprises (i) at least one modified nucleotide that blocks the 5′ OH or phosphate at the 5′ terminus of the passenger strand, wherein the at least one nucleotide modification is an NH₂, biotin, an amine group, a lower alkylamine group, an acetyl group or 2′oxygen-methyl (2′O-Me) modification; or (ii) at least one ribose modification to the active strand or the passenger strand selected from 2′F, 2′NH₂, 2′N₃, 4′thio, or 2′O—CH₃.

In some embodiments, the synthetic miRNA molecule further comprises a complementary strand that is at least 60% complementary to the synthetic miRNA molecule. In some embodiments, the complementary strand is not naturally occurring. In some embodiments, the complementary strand comprises (a) a chemical modification that improves uptake of the synthetic oligonucleotide, (b) a chemical modification that enhances activity of the synthetic oligonucleotide, (c) a chemical modification that enhances stability of the synthetic oligonucleotide, (d) a chemical modification that inhibits uptake of the complementary strand, (e) a chemical modification that inhibits activity of the complementary strand. In some embodiments, the complementary strand comprises one or more nucleobases that are non-complementary with the synthetic miRNA molecule.

c-Met Inhibitors

Disclosed herein are combinations of c-Met inhibitors and a synthetic microRNA molecule comprising (a) an active strand comprising a sequence at least 80% identical to a mature miRNA; and (b) a separate complementary strand that is at least 60% complementary to the active strand. In some embodiments, the c-Met inhibitor is ARQ197 (Tivantinib). In some embodiments, the c-Met inhibitor is EMD1214063 (MSC2156119J; Tepotinib). In some embodiments, the active strand comprises a sequence at least 80% identical to miR-34a.

Further disclosed herein, are methods of treating a cancer in an individual in need thereof comprising administering to the individual a c-Met inhibitor and a synthetic miRNA molecule comprising (a) an active strand comprising a sequence at least 80% identical to a mature miRNA; and (b) a separate complementary strand that is at least 60% complementary to the active strand. In some embodiments, the c-Met inhibitor is ARQ197 (Tivantinib). In some embodiments, the c-Met inhibitor is EMD1214063 (MSC2156119J; Tepotinib). In some embodiments, the active strand comprises a sequence at least 80% identical to miR-34a. In some embodiments, the cancer is selected from the group consisting of: pancreatic, gastric, lung, thyroid, brain, kidney, head and neck, and liver cancer.

c-Met inhibitors are a class of small molecules that inhibit the enzymatic activity of the c-Met tyrosine kinase. These inhibitors may have therapeutic application in the treatment of various types of cancers. c-Met stimulates cell scattering, invasion, protection from apoptosis and angiogenesis. c-Met is a receptor tyrosine kinase, which are implicated in a wide variety of different cancers, such as renal, gastric and small cell lung carcinomas, central nervous system tumors, as well as several sarcomas when its activity is dysregulated. Targeting the ATP binding site of c-Met by small molecules inhibitors is one strategy for inhibition of the tyrosine kinase.

In some embodiments, (i) the c-Met inhibitor selectively binds and inhibits MET kinase (e.g., selectively bind and inhibit dephosphorylated MET kinase); (ii) the c-Met inhibitor is non-ATP competitive inhibitor of MET kinase; and/or (iii) the c-Met inhibitor has cytotoxic activity that is independent from its ability to bind MET kinase. In some embodiments, the c-Met inhibitor is all of (i)-(iii).

Examples of small molecule c-Met inhibitors are provided below. The methods disclosed herein encompass each of these compounds, as well as pharmaceutically acceptable salts and derivatives thereof.

In some embodiments, the c-Met inhibitor is ARQ197 (Tivantinib). In some embodiments, the c-Met inhibitor is EMD1214063 (MSC2156119J; Tepotinib). In some embodiments, the c-Met inhibitor is GSK/1363089/XL880 (Foretinib). In some embodiments, the c-Met inhibitor is XL184 (Cabozantinib). In some embodiments, the c-Met inhibitor is HMPL-504/AZD6094/volitinib (Savolitinib). In some embodiments, the c-Met inhibitor is SOMG-833. In some embodiments, the c-Met inhibitor is MSC2156119J (EMD 1214063, Tepotinib). In some embodiments, the c-Met inhibitor is LY2801653 (Merestinib). In some embodiments, the c-Met inhibitor is AMG 337. In some embodiments, the c-Met inhibitor is INCB28060 (Capmatinib). In some embodiments, the c-Met inhibitor is AMG 458. In some embodiments, the c-Met inhibitor is PF-04217903. In some embodiments, the c-Met inhibitor is PF-02341066 (Crizotinib). In some embodiments, the c-Met inhibitor is E7050 (Golvatinib). In some embodiments, the c-Met inhibitor is MK-2461. In some embodiments, the c-Met inhibitor is BMS-777607. In some embodiments, the c-Met inhibitor is JNJ-38877605. In some embodiments, the c-Met inhibitor is a pyrroloquinolinyl-pyrrolidine-2,5-dione compound of formula IVa, IVb, Va, or Vb, or pharmaceutically acceptable salts thereof. In some embodiments, the c-Met inhibitor is a pyrroloquinolinyl-pyrrolidine-2,5-dione compound of formula IVa, IVb, Va, or Vb, or pharmaceutically acceptable salts thereof.

In some embodiments, the c-Met inhibitor is ARQ197 (Tivantinib). Tivantinib has the IUPAC name (3R,4R)-3-(5,6-Dihydro-4H-pyrrolo[3,2,1-ij]quinolin-1-yl)-4-(1H-indol-3-yl)-2,5-pyrrolidinedione and the following chemical structure:

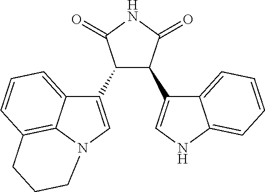 [SMILES: O=C1NC(=O)[C@@H](c2cn3c4c(cccc24)CCC3)[C@@H]1c1c[nH]c2ccccc12]

In some embodiments, the c-Met inhibitor is EMD1214063 (MSC2156119J; Tepotinib). Tepotinib has the IUPAC name 3-(1-(3-(5-((1-methylpiperidin-4-yl)methoxy)pyrimidin-2-yl)benzyl)-1,6-dihydro-6-oxopyridazin-3-yl)benzonitrile and the following chemical structure:

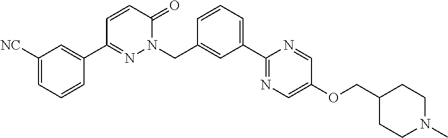 [SMILES: CN1CCC(COc2cnc(-c3cccc(Cn4nc(-c5cccc(C#N)c5)ccc4=O)c3)nc2)CC1]

In some embodiments, the c-Met inhibitor is GSK/1363089/XL880 (Foretinib). Foretinib has the IUPAC name N1′-[3-fluoro-4-[[6-methoxy-7-(3-morpholinopropoxy)-4-quinolyl]oxy]phenyl]-N1-(4-fluorophenyl)cyclopropane-1,1-dicarboxamide and the following chemical structure:

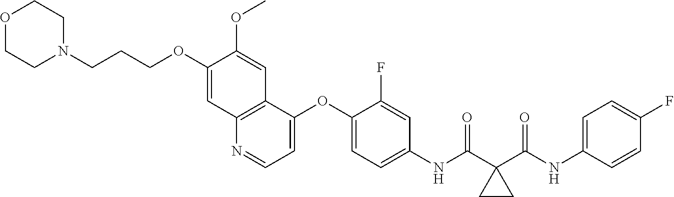 [SMILES: COc1cc2c(Oc3ccc(NC(=O)C4(C(=O)Nc5ccc(F)cc5)CC4)cc3F)ccnc2cc1OCCCN1CCOCC1]

In some embodiments, the c-Met inhibitor is XL184 (Cabozantinib). Cabozantinib has the IUPAC name N-(4-((6,7-Dimethoxyquinolin-4-yl)oxy)phenyl)-N-(4-fluorophenyl)cyclopropane-1,1-dicarboxamide and the following chemical structure:

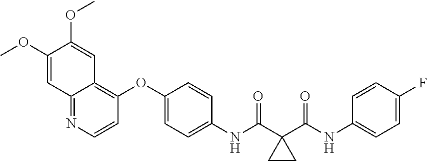 [SMILES: COc1cc2nccc(Oc3ccc(NC(=O)C4(C(=O)Nc5ccc(F)cc5)CC4)cc3)c2cc1OC]

In some embodiments, the c-Met inhibitor is HMPL-504/AZD6094/volitinib (Savolitinib). Volitinib has the IUPAC name (S)-1-(1-(imidazo[1,2-a]pyridin-6-yl)ethyl)-6-(1-methyl-1H-pyrazol-4-yl)-1H-[1,2,3]triazolo[4,5-b]pyrazine and the following chemical structure:

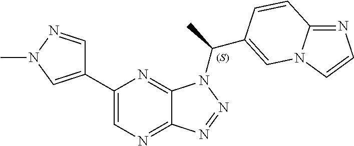

In some embodiments, the c-Met inhibitor is MSC2156119J (EMD 1214063, Tepotinib). Tepotinib has the IUPAC name Benzonitrile, 3-[1,6-dihydro-1-[[3-[5-[(1-methyl-4-piperidinyl)methoxy]-2-pyrimidinyl]phenyl]methyl]-6-oxo-3-pyridazinyl]- and the following chemical structure:

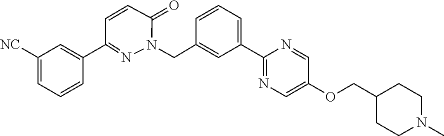 [SMILES: CN1CCC(COc2cnc(-c3cccc(Cn4nc(-c5cccc(C#N)c5)ccc4=O)c3)nc2)CC1]

In some embodiments, the c-Met inhibitor is LY2801653 (Merestinib). Merestinib has the IUPAC name N-(3-fluoro-4-{[1-methyl-6-(1H-pyrazol-4-yl)-1H-indazol-5 yl]oxy}phenyl)-1-(4-fluorophenyl)-6-methyl-2-oxo-1,2-dihydropyridine-3-carboxamide and the following chemical structure:

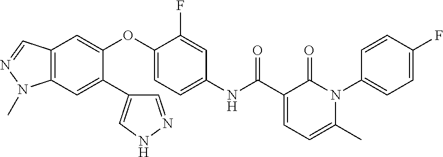 [SMILES: Cc1ccc(C(=O)Nc2ccc(Oc3cc4cnn(C)c4cc3-c3cn[nH]c3)c(F)c2)c(=O)n1-c1ccc(F)cc1]

In some embodiments, the c-Met inhibitor is AMG 337. AMG 337 has the IUPAC name 7-methoxy-N-((6-(3-methylisothiazol-5-yl)-[1,2,4]triazolo[4,3-b]pyridazin-3-yl)methyl)-1,5-naphthyridin-4-amine and the following chemical structure:

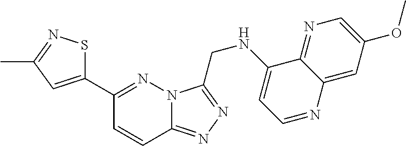

In some embodiments, the c-Met inhibitor is INCB28060 (Capmatinib). Capmatinib has the IUPAC name 2-fluoro-N-methyl-4-[7-(quinolin-6-ylmethyl)imidazo[1,2-b][1,2,4]triazin-2-yl]benzamide and the following chemical structure:

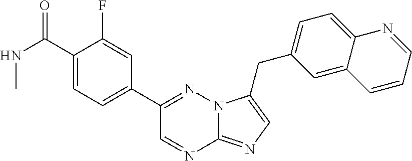 [SMILES: CNC(=O)c1ccc(-c2cnc3ncc(Cc4ccc5ncccc5c4)n3n2)cc1F]

In some embodiments, the c-Met inhibitor is AMG 458. AMG 458 has the IUPAC name 1-(2-hydroxy-2-methylpropyl)-N-(5-((7-methoxyquinolin-4-yl)oxy)pyridin-2-yl)-5-methyl-3-oxo-2-phenyl-2,3-dihydro-1H-pyrazole-4-carboxamide and the following chemical structure:

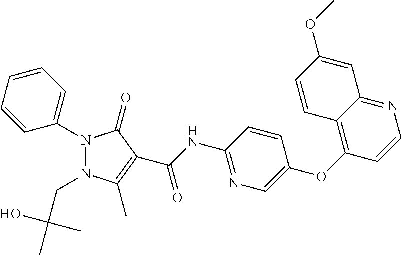 [SMILES: COc1ccc2c(Oc3ccc(NC(=O)c4c(C)n(CC(C)(C)O)n(-c5ccccc5)c4=O)nc3)ccnc2c1]

In some embodiments, the c-Met inhibitor is PF-04217903. PF-04217903 has the IUPAC name 2-(4-(1-(quinolin-6-ylmethyl)-1H-[1,2,3]triazolo[4,5-b]pyrazin-6-yl)-1H-pyrazol-1-yl)ethanol and the following chemical structure:

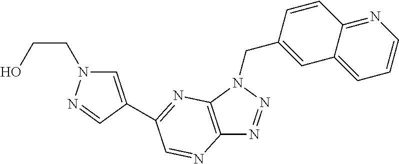 [SMILES: OCCn1cc(-c2cnc3nnn(Cc4ccc5ncccc5c4)c3n2)cn1]

In some embodiments, the c-Met inhibitor is PF-02341066 (Crizotinib). Crizotinib has the IUPAC name (R)-3-(1-(2,6-dichloro-3-fluorophenyl)ethoxy)-5-(1-(piperidin-4-yl)-1H-pyrazol-4-yl)pyridin-2-amine and the following chemical structure:

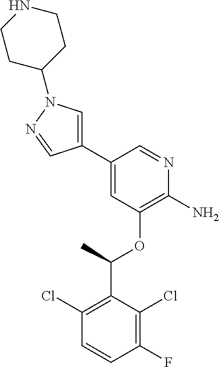 [SMILES: C[C@@H](Oc1cc(-c2cnn(C3CCNCC3)c2)cnc1N)c1c(Cl)ccc(F)c1Cl]

In some embodiments, the c-Met inhibitor is E7050 (Golvatinib). Golvatinib has the IUPAC name N-(2-fluoro-4-((2-(4-(4-methylpiperazin-1-yl)piperidine-1-carboxamido)pyridin-4-yl)oxy)phenyl)-N-(4-fluorophenyl)cyclopropane-1,1-dicarboxamide and the following chemical structure:

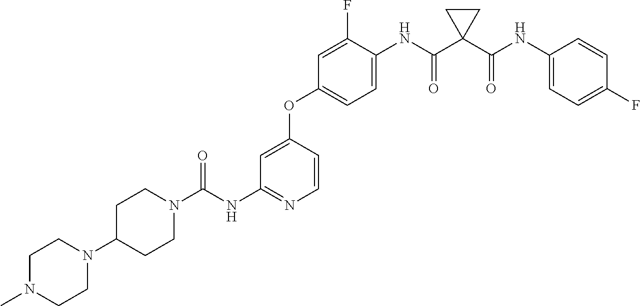 [SMILES: CN1CCN(C2CCN(C(=O)Nc3cc(Oc4ccc(NC(=O)C5(C(=O)Nc6ccc(F)cc6)CC5)c(F)c4)ccn3)CC2)CC1]

In some embodiments, the c-Met inhibitor is MK-2461. MK-2461 has the IUPAC name N-((2R)-1,4-Dioxan-2-ylmethyl)-N-methyl-N′-[3-(1-methyl-1H-pyrazol-4-yl)-5-oxo-5H-benzo[4,5]cyclohepta[1,2-b]pyridin-7-yl]sulfamide and the following chemical structure:

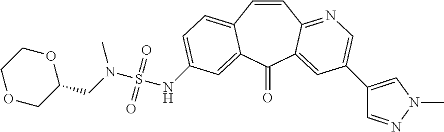 [SMILES: CN(C[C@@H]1COCCO1)S(=O)(=O)Nc1ccc2ccc3ncc(-c4cnn(C)c4)cc3c(=O)c2c1]

In some embodiments, the c-Met inhibitor is BMS-777607. BMS-777607 has the IUPAC name N-(4-((2-amino-3-chloropyridin-4-yl)oxy)-3-fluorophenyl)-4-ethoxy-1-(4-fluorophenyl)-2-oxo-1,2-dihydropyridine-3-carboxamide and the following chemical structure:

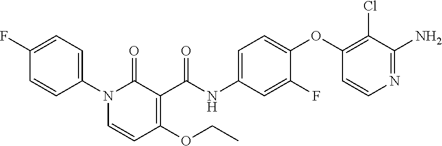 [SMILES: CCOc1ccn(-c2ccc(F)cc2)c(=O)c1C(=O)Nc1ccc(Oc2ccnc(N)c2Cl)c(F)c1]

In some embodiments, the c-Met inhibitor is JNJ-38877605. JNJ-38877605 has the IUPAC name 6-(difluoro(6-(1-methyl-1H-pyrazol-3-yl)-[1,2,4]triazolo[4,3-b]pyridazin-3-yl)methyl)quinoline and the following chemical structure:

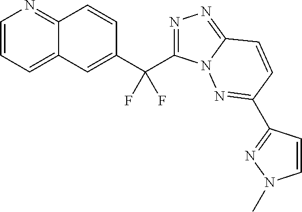 [SMILES: Cn1ccc(-c2ccc3nnc(C(F)(F)c4ccc5ncccc5c4)n3n2)n1]

In some embodiments, the c-Met inhibitor is a pyrroloquinolinyl-pyrrolidine-2,5-dione compound of formula IVa, IVb, Va, or Vb, or pharmaceutically acceptable salts thereof:

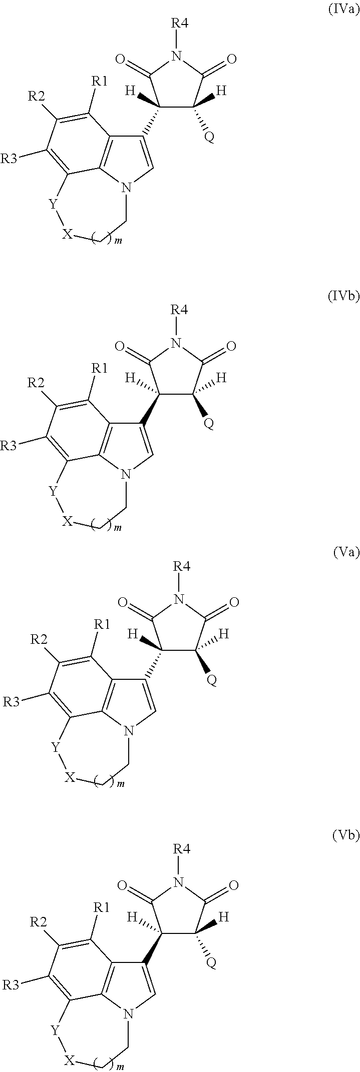

where:

R1, R2 and R3 are independently selected from the group consisting of hydrogen, F, Cl, Br, I, —NR5R6, —(C₁-C₆) alkyl, —(C₁-C₆) substituted alkyl, —(C₃-C₉) cycloalkyl, —(C₃-C₉) substituted cycloalkyl, —O—(C₁-C₆) alkyl, —O—(C₁-C₆) substituted alkyl, —O—(C₃-C₉) cycloalkyl, and —O—(C₃-C₉) substituted cycloalkyl, aryl, heteroaryl, heterocyclyl;

R4 is independently selected from the group consisting of hydrogen, —(C₁-C₆) alkyl, —CH₂R7;

R5, R6 are independently selected from the group consisting of hydrogen, and —(C₁-C₆) alkyl;

R7 is independently selected from the group consisting of —O—P(═O)(OH)₂, —O—P(═O)(—OH)(—O—(C₁-C₆) alkyl), —O—P(═O)(—O—(C₁-C₆) alkyl)₂-O—P(═O)(—OH)(—O—(CH₂)-phenyl), —O—P(═O)(—O—(CH₂)-phenyl)₂, a carboxylic acid group, an amino carboxylic acid group and a peptide;

Q is selected from the group consisting of aryl, heteroaryl, —O-aryl, —S-aryl, —O-heteroaryl, and —S-heteroaryl;

X is selected from the group consisting of —(CH₂)—, —(NR8)-, S, and O;

R8 is independently selected from the group consisting of hydrogen, —(C₁-C₆) alkyl, —(C₁-C₆) substituted alkyl, —(C₃-C₉) cycloalkyl, —(C₃-C₉) substituted cycloalkyl, and —O—(C₁-C₆) alkyl, —C(═O)—O—(C₁-C₆) alkyl and —C(═O)—O—(C₁-C₆) substituted alkyl;

Y is selected from the group consisting of —(CH₂)— or a bond;

wherein said aryl, heteroaryl, —O-aryl, —S-aryl, —O-heteroaryl, and —S-heteroaryl groups may be substituted with one or more substituents independently selected from the group consisting of F, Cl, Br, I, —NR5R6, —(C₁-C₆) alkyl, —(C₁-C₆) substituted alkyl, —(C₃-C₉) cycloalkyl, —(C₃-C₉) substituted cycloalkyl, —O—(C₁-C₆) alkyl, —O—(C₁-C₆) substituted alkyl, —O—(C₃-C₉) cycloalkyl, —O—(C₃-C₉) substituted cycloalkyl, -aryl, -aryl-(C₁-C₆) alkyl, -aryl-O—(C₁-C₆) alkyl, —O-aryl, —O—(C₁-C₄) alkyl-aryl, heteroaryl, heterocyclyl, —O—(C₁-C₄) alkyl-heterocycle, and —(S(O)₂)—(C₁-C₆) alkyl; and

m is 1 or 2.

In some embodiments, R4 is —CH₂R7, and R7 is —O—P(═O)(OH)₂, —O—P(═O)(—OH)(—O—(C₁-C₆) alkyl), —O—P(═O)(—O—(C₁-C₆) alkyl)₂, a carboxylic acid group, an amino carboxylic acid group or a peptide.

In some embodiments, X is selected from the group consisting of —(NR8)-, S, and O.

In some embodiments, m is 2.

In some embodiments, the compound is selected from the group consisting of (+)-cis-3-(5,6-dihydro-4H-pyrrolo[3,2,1-ij]quinolin-lyl)-4(1H-indol-3-yl)pyrrolidine-2,5-dione, (−)-cis-3-(5,6-dihydro-4H-pyrrolo[3,2,1-ᵢⱼ]qumolin-lyl)-4(1H-indol-3-yl)pyrrolidine-2,5-dione, (+)-trans-3-(5,6-dihydro-4H-pyrrolo[3,2,1-ij]quinolin-1-yl)-4(1H-indol-3-yl)pyrrolidine-2,5-dione, and (−)-trans-3-(5,6-dihydro-4H-pyrrolo[3,2,1-ij]quinolin-1-yl)-4(1H-indol-3-yl)pyrrolidine-2,5-dione.

In some embodiments, the compound is (−)-trans-3-(5,6-dihydro-4H-pyrrolo[3,2,1-ij]quinolin-1-yl)-4(1H-indol-3-yl)pyrrolidine-2,5-dione.

In some embodiments, the c-Met inhibitor comprises a class of c-Met inhibitors that function essentially like tivantinib with respect to selectivity, and binding competitiveness, for MET kinase. In some embodiments, the class of c-Met inhibitors also functions essentially like tivantinib with respect to their cytotoxic activity that is independent from its ability to bind MET kinase.

In some embodiments, cancer therapy includes not only the c-Met inhibitors listed above, but also pharmaceutically acceptable salts, isomers, homolog, or analog thereof.

### Methods of Treating Cancers

Disclosed herein, are methods of treating a cancer in an individual in need thereof comprising administering to the individual a c-Met inhibitor and a synthetic miRNA molecule comprising (a) an active strand comprising a sequence at least 80% identical to a mature miRNA; and (b) a separate complementary strand that is at least 60% complementary to the active strand. In some embodiments, the c-Met inhibitor is ARQ197 (Tivantinib). In some embodiments, the c-Met inhibitor is EMD1214063 (MSC2156119J; Tepotinib). In some embodiments, the active strand comprises a sequence at least 80% identical to miR-34a. In some embodiments, the cancer is selected from the group consisting of: pancreatic, gastric, lung, thyroid, brain, kidney, head and neck, and liver cancer.

In some embodiments, combinations of a c-Met inhibitor and a synthetic miRNA molecule are effective at inhibiting the proliferation of cancer cells. In some embodiments, combinations of a c-Met inhibitor and a synthetic miRNA molecule are effective at preventing the proliferation of cancer cells.

In some embodiments, combinations a c-Met inhibitor and a synthetic miRNA molecule have increased efficacy as compared to administration of a c-Met inhibitor or synthetic miRNA molecule alone. In some embodiments, combinations a c-Met inhibitor and a synthetic miRNA molecule is synergistic. In some embodiments, combinations a c-Met inhibitor and a synthetic miRNA molecule reduces toxicity associated with the c-Met inhibitor or the synthetic miRNA molecule.

In some embodiments, the subject is a primate, such as a human, with liver cancer. Examples of mammal include, but are not limited to, murines, simians, humans, farm animals, sport animals, and pets. In some embodiments, the subject is an adult human (i.e., 18 years or older). In some embodiments, the subject is a juvenile human (i.e., less than 18 years old).

In some embodiments, methods are applicable to the treatment of cancer cells, including cancer cells in a subject or in vitro treatment of isolated cancer cells.

In some embodiments, the cancer (e.g., liver cancer such as HCC) is not resistant to the c-Met inhibitor. In some embodiments, the cancer (e.g., liver cancer such as HCC) is not resistant to tivantinib. In some embodiments, the cancer (e.g., liver cancer such as HCC) is not resistant to Tepotinib. In some embodiments, the subject is a responder to the c-Met inhibitor in the absence of the synthetic miRNA molecule. In some embodiments, the subjects are patients who have experienced one or more significant adverse side effect to the c-Met inhibitor. In some embodiments, administration of the synthetic miRNA molecule and the c-Met inhibitor results in a decreased dosage of the c-Met inhibitor. In some embodiments, administration of the synthetic miRNA molecule and the c-Met inhibitor results in a decreased dosage of the synthetic miRNA molecule.

In some embodiments, the cancer has primary or secondary resistance to the c-Met inhibitor. In some embodiments, the cancer (e.g., liver cancer such as HCC) has primary or secondary resistance to tivantinib. In some embodiments, the cancer (e.g., liver cancer such as HCC) has primary or secondary resistance to Tepotinib. In some embodiments, the methods disclosed herein further comprise determining whether the individual has resistance to the c-Met inhibitor. In some embodiments, the subject is a non-responder to the c-Met inhibitor in the absence of the synthetic miRNA molecule. Disclosed herein, are methods of treating a cancer with resistance to a c-Met inhibitor in an individual in need thereof comprising (a) identifying the cancer as a cancer with resistance to treatment with a c-Met inhibitor; and (b) administering to the individual (i) a c-Met inhibitor and (ii) a synthetic miRNA molecule comprising (A) an active strand comprising a sequence at least 80% identical to a mature miRNA; and (B) a separate complementary strand that is at least 60% complementary to the active strand. In some embodiments, the c-Met inhibitor is ARQ197 (Tivantinib). In some embodiments, the c-Met inhibitor is EMD1214063 (MSC2156119J; Tepotinib). In some embodiments, the active strand comprises a sequence at least 80% identical to miR-34a. In some embodiments, the cancer is selected from the group consisting of: pancreatic, gastric, lung, thyroid, brain, kidney, head and neck, and liver cancer.

In some embodiments, the subject has undergone a prior treatment with the c-Met inhibitor lasting at least 2, 4, 6, 8, 10 months or longer.

In some embodiments, the subjects are patients who have experienced one or more significant adverse side effect to the c-Met inhibitor.

In some embodiments, the cancer is intermediate, advanced, or terminal stage. In some embodiments, the cancer is metastatic. In some embodiments, the cancer is non-metastatic.

### Liver Cancer

Disclosed herein are methods of treating a liver cancer in an individual in need thereof comprising administering to the individual a c-Met inhibitor and a synthetic miRNA molecule comprising (a) an active strand comprising a sequence at least 80% identical to a mature miRNA; and (b) a separate complementary strand that is at least 60% complementary to the active strand. In some embodiments, the c-Met inhibitor is ARQ197 (Tivantinib). In some embodiments, the c-Met inhibitor is EMD1214063 (MSC2156119J; Tepotinib). In some embodiments, the active strand comprises a sequence at least 80% identical to miR-34a.

In some embodiments, the liver cancer is primary liver cancer. In some embodiments, the liver cancer is HCC.

In some embodiments, the liver cancer (e.g., HCC) is resectable. In some embodiments, the liver cancer (e.g., HCC) is unresectable. In some embodiments, the cancer comprises a single tumor, multiple tumors, or a poorly defined tumor with an infiltrative growth pattern (into portal veins or hepatic veins in the case of liver cancer). In some embodiments, the liver cancer comprises a fibrolamellar, pseudoglandular (adenoid), pleomorphic (giant cell), or clear cell pattern. In some embodiments, liver cancer (e.g., HCC) comprises a well differentiated form, and tumor cells resemble hepatocytes, form trabeculae, cords, and nests, and/or contain bile pigment in cytoplasm. In some embodiments, liver cancer (e.g., HCC) comprises a poorly differentiated form, and malignant epithelial cells are discohesive, pleomorphic, anaplastic, and/or giant. In some embodiments, the liver cancer (e.g., HCC) is associated with hepatitis B, hepatitis C, cirrhosis, or type 2 diabetes.

Liver cancer (or hepatic cancer) is a cancer that originates in the liver. Primary liver cancer is the fifth most frequently diagnosed cancer globally and the second leading cause of cancer death. Liver cancers are malignant tumors that grow on the surface or inside the liver. They are formed from either the liver itself or from structures within the liver, including blood vessels or the bile duct.

The leading cause of liver cancer is viral infection with hepatitis B virus or hepatitis C virus. The cancer usually forms secondary to cirrhosis caused by these viruses. For this reason, the highest rates of liver cancer occur where these viruses are endemic, including East-Asia and sub-Saharan Africa.

The most frequent liver cancer, accounting for approximately 75% of all primary liver cancers, is hepatocellular carcinoma (HCC). HCC is a cancer formed by liver cells, known as hepatocytes that become malignant. Another type of cancer formed by liver cells is hepatoblastoma, which is specifically formed by immature liver cells. It is a rare malignant tumor that primarily develops in children, and accounts for approximately 1% of all cancers in children and 79% of all primary liver cancers under the age of 15.

Liver cancer can also form from other structures within the liver such as the bile duct, blood vessels and immune cells. Cancer of the bile duct (cholangiocarcinoma and cholangiocellular cystadenocarcinoma) account for approximately 6% of primary liver cancers. There is also a variant type of HCC that consists of both HCC and cholangiocarcinoma. Tumors of the liver blood vessels include angiosarcoma and hemangioendothelioma. Embryonal sarcoma and fibrosarcoma are produced from a type of connective tissue known as mesenchyme. Cancers produced from muscle in the liver are leiomyosarcoma and rhabdomyosarcoma. Other less common liver cancers include carcinosarcomas, teratomas, yolk sac tumors, carcinoid tumors and lymphomas. Lymphomas usually have diffuse infiltration to liver, but it may also form a liver mass in rare occasions.

Surgical resection is often the treatment of choice for non-cirrhotic livers. Increased risk of complications such as liver failure can occur with resection of cirrhotic livers. 5-year survival rates after resection has massively improved over the last few decades and can now exceed 50%. Recurrence rates after resection due to the spread of the initial tumor or formation of new tumors exceeds 70%. Liver transplantation can also be used in cases of HCC where this form of treatment can be tolerated and the tumor fits specific criteria (e.g., the Milan criteria). Less than 30-40% of individuals with HCC are eligible for surgery and transplant because the cancer is often detected at a late stage. Also, HCC can progress during the waiting time for liver transplants, which can ultimately prevent a transplant.

Percutaneous ablation is the only non-surgical treatment that can offer cure. There are many forms of percutaneous ablation, which consist of either injecting chemicals into the liver (ethanol or acetic acid) or producing extremes of temperature using radio frequency ablation, microwaves, lasers or cryotherapy. Of these, radio frequency ablation has a relatively positive record of treating HCC, but the limitations include inability to treat tumors close to other organs and blood vessels due to heat generation and the heat sync effect, respectively.

Systemic chemotherapeutics are not routinely used in HCC, although local chemotherapy may be used in a procedure known as transarterial chemoembolization. In this procedure, cytotoxic drugs such as doxorubicin or cisplatin with lipiodol are administered and the arteries supplying the liver are blocked by gelatin sponge or other particles. Subjects undergoing chemotherapy often suffer toxic side effects such as nausea and vomiting, hair loss, loss of appetite and increased chances of infections, easy injury or bleeding, and fatigue.

Radiotherapy is not often used in HCC because the liver is not tolerant to radiation. Although with modern technology it is possible to provide well targeted radiation to the tumor, minimizing the dose to the rest of the tumor. Dual treatments of radiotherapy plus chemoembolization, local chemotherapy, systemic chemotherapy or targeted therapy drugs may show benefit over radiotherapy alone.

### Formulations and Dosage

Disclosed herein are combinations of c-Met inhibitors and a synthetic microRNA molecule comprising (a) an active strand comprising a sequence at least 80% identical to a mature miRNA; and (b) a separate complementary strand that is at least 60% complementary to the active strand. In some embodiments, the c-Met inhibitor is ARQ197 (Tivantinib). In some embodiments, the c-Met inhibitor is EMD1214063 (MSC2156119J; Tepotinib). In some embodiments, the active strand comprises a sequence at least 80% identical to miR-34a.

Further disclosed herein, are methods of treating a cancer in an individual in need thereof comprising administering to the individual a c-Met inhibitor and a synthetic miRNA molecule comprising (a) an active strand comprising a sequence at least 80% identical to a mature miRNA; and (b) a separate complementary strand that is at least 60% complementary to the active strand. In some embodiments, the c-Met inhibitor is ARQ197 (Tivantinib). In some embodiments, the c-Met inhibitor is EMD1214063 (MSC2156119J; Tepotinib). In some embodiments, the active strand comprises a sequence at least 80% identical to miR-34a. In some embodiments, the cancer is selected from the group consisting of: pancreatic, gastric, lung, thyroid, brain, kidney, head and neck, and liver cancer.

A variety of formulations are available for encapsulating the synthetic microRNA molecules disclosed herein in liposomes. In some embodiments, the microRNA is formulated in amphoteric liposomes, for example Marina Biotech's SMARTICLES®. In some embodiments, amphoteric liposomes comprise one or more (e.g. 1, 2, 3, or 4) of cholesterol hemisuccinate, morpholino cholesterol, POPC, and DOPE. In some embodiments, the liposome formulation is cholesterol-siRNA, RNA aptamers-siRNA, stable nucleic acid lipid particle (SNALP), cardiolipin analog-based liposome, DSPE-polyethylene glycol-DOTAP-cholesterol liposome, hyaluronan-DPPE liposome, neutral DOPC liposome, oligoarginine (9R) conjugated water soluble lipopolymer (WSLP), cholesterol-MPG-8, DOPE-cationic liposome, GALA peptide-PEG-MMP-2 cleavable peptide-DOPE and the like. In some embodiments, the liposome comprises the following lipids: morpholinoethaneamine-cholesterol, cholesteryl hemisuccinate, 1,2-dioleoyl-sn-glycero-3-phosphoethanolamine, and 1-palmitoyl-2-oleoyl-sn-glycero-3-phosphocholine.

In some embodiments, the synthetic miRNA molecule is in a sterile aqueous solution. In some embodiments, the synthetic miRNA molecule is a synthetic and/or non-naturally occurring liposome. In some embodiments, the synthetic miRNA molecule is in a solution that further comprises an antibacterial or antifungal agent. In some embodiments, the synthetic miRNA molecule is at least about 0.1% by weight of the solution. In some embodiments, the synthetic miRNA molecule is at least about 2% to about 75% by weight of the solution. In some embodiments, the synthetic miRNA molecule is at least about 25% to about 60% by weight of the solution. In some embodiments, the synthetic miRNA molecule is at least about 95% pure. In some embodiments, the synthetic miRNA molecule is at least about 96% pure. In some embodiments, the synthetic miRNA molecule is at least about 97% pure. In some embodiments, the synthetic miRNA molecule is at least about 98% pure. In some embodiments, the synthetic miRNA molecule is at least about 99% pure. In some embodiments, the synthetic miRNA molecule is at least about 100% pure. In some embodiments, the synthetic miRNA molecule is in a solution that is aliquoted in a vial, test tube, flask, bottle, syringe, or container. In some embodiments, these and other solutions are formulated for administration to a subject intravenously or by injection. In some embodiments, the synthetic miRNA molecule is a solid, for example lyophilized or in a dry powder.

In some embodiments, the synthetic miRNA molecule is administered in a dose, or in a dosage form, of about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, about 1000 mg/kg body weight, about 5 mg/kg body weight to about 100 mg/kg body weight, or about 5 μg/kg body weight to about 500 mg/kg body weight.

In some embodiments, synthetic miRNA molecule is administered intravenously as a slow-bolus injection at doses ranging 0.001-6.0 mg/kg per dose, for example, 0.01-3.0, 0.025-1.0 or 0.25-0.5 mg/kg per dose, with one, two, three or more doses per week for 2, 4, 6, 8 weeks or longer as necessary.

In some embodiments, the c-Met inhibitor and synthetic miRNA molecule are provided in molar ratio of about 15-3000 or about 320-31250.

In some embodiments, the molar ratio of c-Met inhibitor:synthetic miRNA molecule, based on the amount of c-Met inhibitor and synthetic miRNA molecule provided to the subject in a single day, is about 15-764. In some embodiments, the molar ratio of c-Met inhibitor:synthetic miRNA molecule, based on the amount of c-Met inhibitor and synthetic miRNA molecule provided to the subject over a single week, is about 22-2674. In some embodiments, the molar ratio of c-Met inhibitor:synthetic miRNA molecule, based on the amount of c-Met inhibitor and synthetic miRNA molecule provided to the subject in a single day, is about 15, 21, 27, 31, 36, 41, 51, 55, 62, 73, 77, 82, 93, 102, 110, 123, 127, 146, 154, 164, 204, 218, 255, 306, 309, 463, 509, or 764. In some embodiments, the molar ratio of c-Met inhibitor:synthetic miRNA molecule, based on the amount of c-Met inhibitor and synthetic miRNA molecule administered to the subject over a single week, is about 22, 29, 38, 43, 51, 54, 58, 71, 72, 77, 86, 96, 102, 108, 115, 127, 130, 143, 144, 153, 173, 178, 192, 204, 216, 230, 255, 270, 285, 288, 306, 324, 357, 383, 428, 431, 432, 446, 509, 540, 575, 648, 713, 764, 891, 1070, 1070, 1081, 1621, 1783, or 2674.

In some embodiments, the c-Met inhibitor and synthetic miRNA molecule have a combination index (CI)<1. The combination index (CI) is less than about 0.80, 0.75, 0.70, 0.65, 0.60, 0.55, 0.50, 0.45, 0.40, 0.35, 0.30, 0.25, or 0.20.

Additional ratios and ranges are provided throughout the specification and examples.

In some embodiments, the molar ratio of c-Met inhibitor:synthetic miRNA molecule is measured over different periods of time. In some embodiments, the molar ratio is the amount of c-Met inhibitor:synthetic miRNA molecule administered to the subject in a single day. In some embodiments, the molar ratio is the amount of c-Met inhibitor:synthetic miRNA molecule administered to the subject in a single week. In some embodiments, the molar ratio is the amount of c-Met inhibitor:synthetic miRNA molecule administered to the subject over 14 days. In some embodiments, the molar ratio is the amount of c-Met inhibitor:synthetic miRNA molecule administered to the subject over 21 days. In some embodiments, the molar ratio is the amount of c-Met inhibitor:synthetic miRNA molecule administered to the subject over 28 days.

In some embodiments, c-Met inhibitor dosing amount and/or schedule follows clinically approved, or experimental, guidelines. In some embodiments, the dose of c-Met inhibitor, such as tivantinib, is about 720, 480, 240, or 120 mg/day. In some embodiments, other dosing such as 800, 600, 400, or 200 mg/day is possible. In some embodiments, doses are grouped and given on alternating days—for example, a 200 mg/day dose is administered as a 400 mg dose every other day.

In some embodiments, effective dosages achieved in one animal are extrapolated for use in another animal, including humans, using conversion factors as exemplified in Table 2.

TABLE 2
Equivalent Surface Area Dosage Factors

From:

| | Mouse | Rat | Monkey | Dog | Human |
|---|---|---|---|---|---|
| To: | (20 g) | (150 g) | (3.5 kg) | (8 kg) | (60 kg) |
| Mouse | 1 | 0.5 | 0.25 | 0.17 | 0.08 |
| Rat | 2 | 1 | 0.5 | 0.25 | 0.14 |
| Monkey | 4 | 2 | 1 | 0.6 | 0.33 |
| Dog | 6 | 4 | 1.7 | 1 | 0.5 |
| Human | 12 | 7 | 3 | 2 | 1 |

In some embodiments, synthetic miRNA molecule dosing amount and/or schedule follows clinically approved, or experimental, guidelines. In some embodiments, the dose of synthetic miRNA molecule is about 10, 20, 25, 30, 40, 50, 75, 100, 125, 150, 175, 200, 225, or 250 mg/m² per day. In some embodiments, the dose is set, within a therapeutically effective range, based upon a selected ratio and dose of c-Met inhibitor. In some embodiments, the ratio is determined using the amount of synthetic miRNA molecule administered to a subject over a single day, a single week, 14 days, 21 days, or 28 days.

In some embodiments the synthetic miRNA molecule is administered to the subject in 1, 2, 3, 4, 5 daily doses over 5 days. In some embodiments, the synthetic miRNA molecule is administered to the subject in 1, 2, 3, 4, 5, 6, or 7 daily doses over a single week (7 days). In some embodiments, the synthetic miRNA molecule is administered to the subject in 1, 2, 3, 4, 5, 6, 7, 8, 9, 10, 11, 12, 13, or 14 daily doses over 14 days. In some embodiments, the synthetic miRNA molecule is administered to the subject in 1, 2, 3, 4, 5, 6, 7, 8, 9, 10, 11, 12, 13, 14, 15, 16, 17, 18, 19, 20, or 21 daily doses over 21 days. In some embodiments, the synthetic miRNA molecule is administered to the subject in 1, 2, 3, 4, 5, 6, 7, 8, 9, 10, 11, 12, 13, 14, 15, 16, 17, 18, 19, 20, 21, 22, 23, 24, 25, 26, 27, or 28 daily doses over 28 days.

In some embodiments, the synthetic miRNA molecule is administered for 2 weeks (total 14 days). In some embodiments, the synthetic miRNA molecule is administered for 1 week with 1 week off (total 14 days). In some embodiments, the synthetic miRNA molecule is administered for 3 consecutive weeks (total 21 days). In some embodiments, the synthetic miRNA molecule is administered for 2 weeks with 1 week off (total 21 days). In some embodiments, the synthetic miRNA molecule is administered for 1 week with 2 weeks off (total 21 days). In some embodiments, the synthetic miRNA molecule is administered for 4 consecutive weeks (total 28 days). In some embodiments, the synthetic miRNA molecule is administered for 3 consecutive weeks with 1 week off (total 28 days). In some embodiments, the synthetic miRNA molecule is administered for 2 weeks with 2 weeks off (total 28 days). In some embodiments, the synthetic miRNA molecule is administered for 1 week with 3 consecutive weeks off (total 28 days).

In some embodiments, the synthetic miRNA molecule is administered on day 1 of a 7, 14, 21 or 28 day cycle. In some embodiments, the synthetic miRNA molecule is administered on days 1 and 15 of a 21 or 28 day cycle. In some embodiments, the synthetic miRNA molecule is administered on days 1, 8, and 15 of a 21 or 28 day cycle. In some embodiments, the synthetic miRNA molecule is administered on days 1, 2, 8, and 15 of a 21 or 28 day cycle. In some embodiments, the synthetic miRNA molecule is administered once every 1, 2, 3, 4, 5, 6, 7, or 8 weeks.

In some embodiments, the synthetic miRNA molecule is administered once a day for three days in a 7 day period (QDx3), once a day for four days in a 7 day period (QDx4), or once a day for five days in a 7 day period QDx5. In some embodiments, the synthetic miRNA molecule is administered QDx3, QDx4, or QDx5 for 3 weeks. In some embodiments, the synthetic miRNA molecule is administered QDx5. In some embodiments, the synthetic miRNA molecule is administered QDx5 for 3 weeks. In some embodiments, the synthetic miRNA molecule is administered once a day for two days in a 7 day period (BIW). In some embodiments, the synthetic miRNA molecule is administered BIW for 3 weeks.

In some embodiments, a course of c-Met inhibitor-synthetic miRNA molecule combination therapy is prescribed by a clinician. In some embodiments, the synthetic miRNA molecule (and hence the combination therapy) is administered for 1, 2, 3, 4, 5, 6, 7, 8, 9, 10, 11, or 12 cycles.

In some embodiments, a course of c-Met inhibitor-synthetic miRNA molecule combination therapy is continued until a clinical endpoint is met. In some embodiments, the therapy is continued until disease progression or unacceptable toxicity occurs. In some embodiments, the therapy is continued until achieving a pathological complete response (pCR) rate defined as the absence of liver cancer (e.g., HCC). In some embodiments, the therapy is continued until partial or complete remission of the liver cancer. In some embodiments, administering the synthetic miRNA molecule and a c-Met inhibitor to a plurality of subject having HCC increases the Overall Survival (OS), the Progression free Survival (PFS), the Disease Free Survival (DFS), the Response Rate (RR), the Quality of Life (QoL), or a combination thereof.

In some embodiments, the treatment reduces the size and/or number of the cancer tumor(s). In some embodiments, the treatment prevents the cancer tumor(s) from increasing in size and/or number. In some embodiments, the treatment prevents the cancer tumor(s) from metastasizing.

In some embodiments, are methods of administration which are not limited to any particular delivery system and may include, without limitation, parenteral (including subcutaneous, intravenous, intramedullary, intraarticular, intramuscular, or intraperitoneal injection), rectal, topical, transdermal, or oral (for example, in capsules, suspensions, or tablets). In some embodiments, administration to an individual occurs in a single dose or in repeat administrations. In some embodiments, administration to an individual occurs in any of a variety of physiologically acceptable salt forms, and/or with an acceptable pharmaceutical carrier and/or additive as part of a pharmaceutical composition.

In some embodiments, the synthetic miRNA molecule is administered prior to the c-Met inhibitor. In some embodiments, the synthetic miRNA molecule is administered concurrently with the c-Met inhibitor. In some embodiments, the synthetic miRNA molecule is administered after the c-Met inhibitor.

In some embodiments, the synthetic miRNA molecule is administered intravenously. In some embodiments, the synthetic miRNA molecule is administered systemically or regionally.

In some embodiments, the therapeutically effective dose of c-Met inhibitor is reduced through combination with the synthetic miRNA molecule. For example, the weekly or monthly dose of c-Met inhibitor can be reduced by at least 10%, 20%, 30%, 40%, 50%, 60%, 70%, 80%, 90% or more relative to the maximum recommended dose or the maximum tolerated dose.

In some embodiments, the c-Met inhibitor is administered at an effective dose that at least 50% or more below the dose needed to be effective in the absence of the synthetic miRNA molecule administration. In some embodiments, the c-Met inhibitor is administered at an effective dose that at least 60% or more below the dose needed to be effective in the absence of the synthetic miRNA molecule administration. In some embodiments, the c-Met inhibitor is administered at an effective dose that at least 70% or more below the dose needed to be effective in the absence of the synthetic miRNA molecule administration. In some embodiments, the c-Met inhibitor is administered at an effective dose that at least 80% or more below the dose needed to be effective in the absence of the synthetic miRNA molecule administration. In some embodiments, the c-Met inhibitor is administered at an effective dose that at least 90% or more below the dose needed to be effective in the absence of the synthetic miRNA molecule administration.

In some embodiments, the IC50 of the c-Met inhibitor is reduced by at least 4-fold relative to the IC50 in the absence of the synthetic miRNA molecule. In some embodiments, the IC50 of the c-Met inhibitor is reduced by at least 5-fold relative to the IC50 in the absence of the synthetic miRNA molecule. In some embodiments, the IC50 of the c-Met inhibitor is reduced by at least 10-fold relative to the IC50 in the absence of the synthetic miRNA molecule. In some embodiments, the IC50 of the c-Met inhibitor is reduced by at least 20-fold relative to the IC50 in the absence of the synthetic miRNA molecule. In some embodiments, the IC50 of the c-Met inhibitor is reduced by at least 30-fold relative to the IC50 in the absence of the synthetic miRNA molecule. In some embodiments, the IC50 of the c-Met inhibitor is reduced by at least 40-fold relative to the IC50 in the absence of the synthetic miRNA molecule. In some embodiments, the IC50 of the c-Met inhibitor is reduced by at least 50-fold relative to the IC50 in the absence of the synthetic miRNA molecule. In some embodiments, the IC50 of the c-Met inhibitor is reduced by at least 100-fold relative to the IC50 in the absence of the synthetic miRNA molecule.

### Synergy and Combination Index (CI) Values

Disclosed herein are methods of treating a cancer in an individual in need thereof. In some embodiments, the methods comprise administering to the individual a c-Met inhibitor and a synthetic miRNA molecule. In some embodiments, the methods and compositions comprise a c-Met inhibitor and synthetic miRNA molecule administered in a ratio that is particularly effective (e.g., synergistic or more than additive). In some embodiments, combination index (CI) values are used to quantify the effects of various combinations of c-Met inhibitor and synthetic miRNA molecule.

In some embodiments, CI values are based on Loewe's additivity model to assess the nature of drug-drug interactions that can be additive (CI=1), antagonistic (CI>1), or synergistic (CI<1) for various drug-drug concentrations and effect levels (Fa, fraction affected; inhibition of cancer cell proliferation). CI values are calculated based on linear regression trendlines using the CompuSyn software (ComboSyn Inc., Paramus, N.J.) whereby the hyperbolic and sigmoidal dose-effect curves are transformed into a linear form.

In some embodiments, the molar ratio of c-Met inhibitor:synthetic miRNA molecule exhibits a CI<1. In some embodiments, the molar ratio of c-Met inhibitor:synthetic miRNA molecule has a CI<0.95, 0.90, 0.85, 0.80, 0.75, 0.70, 0.65, 0.60, 0.55, 0.50, 0.45, 0.40, 0.35, 0.30, 0.25, or 0.20. In some embodiments, the synthetic miRNA molecule is miR-34a (e.g., a miR-34 family mimic) and the CI<0.60. In some embodiments, CI is used in conjunction with other parameters, for example CI<0.60, DRI>2, and Fa>65%. In some embodiments, the synthetic miRNA molecule is miR-34 family mimic and the CI<0.80, 0.75, 0.70, 0.65, 0.60, 0.55, or 0.50 (and optionally in combination with other parameters, for example DRI>2, and Fa>65%).

In some embodiments, in the case of human therapy, the CI value is considered to be the CI value of a reference system—for example, a cell assay, e.g., as described herein, or an animal model, e.g., rat or non-human primate.

### Additional Therapies

Disclosed herein are combinations of (a) c-Met inhibitors, (b) a synthetic microRNA molecule comprising (i) an active strand comprising a sequence at least 80% identical to a mature miRNA; and (ii) a separate complementary strand that is at least 60% complementary to the active strand; and (c) an additional therapy. In some embodiments, the c-Met inhibitor is ARQ197 (Tivantinib). In some embodiments, the c-Met inhibitor is EMD1214063 (MSC2156119J; Tepotinib). In some embodiments, the active strand comprises a sequence at least 80% identical to miR-34a.

Further disclosed herein, are methods of treating a cancer in an individual in need thereof comprising administering to the individual (a) a c-Met inhibitor; (b) a synthetic miRNA molecule comprising (i) an active strand comprising a sequence at least 80% identical to a mature miRNA; and (ii) a separate complementary strand that is at least 60% complementary to the active strand; and (c) an additional therapy. In some embodiments, the c-Met inhibitor is ARQ197 (Tivantinib). In some embodiments, the c-Met inhibitor is EMD1214063 (MSC2156119J; Tepotinib). In some embodiments, the active strand comprises a sequence at least 80% identical to miR-34a. In some embodiments, the cancer is selected from the group consisting of: pancreatic, gastric, lung, thyroid, brain, kidney, head and neck, and liver cancer.

In some embodiments, the additional therapy is surgical resection, percutaneous ethanol or acetic acid injection, transcatheter arterial chemoembolization, radiofrequency ablation, laser ablation, cryoablation, focused external beam radiation stereotactic radiotherapy, selective internal radiation therapy, intra-arterial iodine-131-lipiodol administration, and/or high intensity focused ultrasound.

In some embodiments, the additional therapy is a chemotherapeutic agent. Any suitable chemotherapeutic agent may be used in combination with the c-Met inhibitor and the synthetic miRNA. In some embodiments, the additional therapy is dexamethasone.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### C-Met Inhibitor and miR-Rx34 Combination Studies in Liver Cancer Cells

Summary:

Tivantinib is an orally bioavailable small molecule inhibitor of c-Met with potential antineoplastic activity. It is currently in Phase III study to treat patients with hepatocellular carcinoma (HCC). EMD1214063 is a highly selective, reversible, ATP-competitive c-Met inhibitor that causes growth inhibition, and regression of hepatocyte growth factor-dependent and -independent tumors in preclinical models. It is currently in Phase I study to treat patients with hepatocellular carcinoma (HCC) with active c-Met signaling. Therapeutic miRNA mimics modeled after endogenous tumor suppressor miRNAs inhibit tumor growth by repressing multiple oncogenes at once and, therefore, may be used to augment drug sensitivity. Here, we investigated the relationship of miR-34a mimics (miR-Rx34) and c-Met inhibitors and determined the therapeutic activity of the combination in a panel of human HCC cell lines. Using multiple analytical approaches, drug-induced inhibition of cancer cell proliferation was determined to reveal additive, antagonistic or synergistic effects. The data showed a synergistic interaction between tivantinib and miR-34a mimics, as well as EMD 1214063 and miR-34a mimics in various HCC cells.

Materials and Reagents:

miRNAs: miR-Rx34 (Ambion, Cat#AM16099, Lot#AS00012XE); alias: miR-34a. The miRNA manufactures are in-vivo ready quality and prepared as a 600 nM stock solution in nuclease-free H2O. Tivantinib: 10 mM in DMSO (Selleckchem.com, Cat#52753, CAS#905854-02-6). EMD1214063: 10 mM in DMSO (Selleckchem.com, Cat#57067, CAS#1100598-32-0). Cell Lines: Hep3b, HepG2, C3A (ATCC HB-8064, HB-8065, CRL-10741, HTB-52) and Huh7 (Japanese Collection of Research Bioresources Cell Bank). Cell culture medium: EMEM (ATCC, Cat#30-2003, Lot#60946371); DMEM (Gibco, Cat#11320-033, Lot#1147373); Trypsin (Gibco, Cat#25300-054); PBS (Ambion, Cat#AM9625); Opti-MEM (Gibco, Cat#31985-070, Lot#1293625); Lipofectamine RNAiMAX transfection reagent (Life Technology, Cat#13778-150, Lot#1233863); and AlamarBlue (Life Technology, Cat#DAL1100, Lot#156129SA). Instruments: PolarStar Optima plate reader (BMG Labtech).

Experimental Procedures:

Cell culture: HCC cells were maintained in EMEM medium with 10% fetal bovine serum, except Huh7 were maintained in DMEM. All cells were maintained at 37° C. in a humidified of 95% air and 5% CO₂.

miR-Rx34 and tivantinib and EMD1214063 treatment: To determine the IC50 value of each drug alone, 2,000 cells per well were seeded in a 96-well plate format and treated with either tivantinib or miR-Rx34 as follows. (i) miR-Rx34 was reverse-transfected in triplicates in a serial dilution (0.03-30 nM) using RNAiMax lipofectamine. As controls, cells were also transfected with RNAiMax alone (mock). Cells were incubated with AlamarBlue (Invitrogen) 6 days post transfection to determine cellular proliferation. Proliferation data were normalized to mock-transfected cells. (ii) Tivantinib or EMD1214063, prepared as a 10 mM stock solution in dimethyl sulfoxide (DMSO), was added to cells 3 days after seeding at a final concentration ranging from 0.1 to 100 μM for tivantinib and 0.01 and 10 μM for EMD1214063. Solvent alone (1% final DMSO) was added to cells in separate wells as a control. Three days thereafter, cellular proliferation was measured by AlamarBlue and normalized to the solvent control.

Combination effects determined by the Fixed Ratio method: The combination studies were carried out at ˜IC50 ratio of c-Met inhibitor and miR-Rx34 (ratio=IC50 c-Met inhibitor/IC50 miR-Rx34). Cells were treated with tivantinib or EMD1214063 in combination with miR-Rx34a at a concentration approximately equal to its corresponding IC50 and concentrations within 2-fold or 2.5-fold increments above or below. The ratios of tivantinib/miR-Rx34a are 12500 in C3A, 3750 in Hep3b, 4000 in HepG2 and 1500 in Huh7. The ratios of EMD1214063/miR-Rx34a are 333.3 in SK-HEP1 and 3333.3 in C3A. Cells were reverse transfected with miR-Rx34a, and c-Met inhibitor was added 3 days post transfection. Cell proliferation was measured 3 days post c-Met inhibitor addition by AlamarBlue. Each data points were done in triplicates, and the combination studies were repeated three times in each cell line.

The tivantinib combination studies were also carried out at multiple ratios in Hep3B and HepG2 cells. Cells were treated with 7 concentrations of tivantinib each in combination with 7 concentrations of miR Rx34. Each drug was used at a concentration approximately equal to its IC50 and at concentrations within 2.5-fold increments above or below. This matrix yielded a total of 49 different combinations representing 13 different ratios. Each drug was also used alone at these concentrations. Cells were reversed transfected with miR-Rx34 and incubated for 3 days until tivantinib was added to the medium. Another 3 days later, cellular proliferation was determined by AlamarBlue. Each data point was performed in triplicates, and the combination studies were repeated three times in each cell line. The EMD1214063 combination studies were carried out at multiple ratios in C3A and SK-HEP1 cells. Cells were treated with 7 concentrations of EMD 1214063 each in combination with 7 concentrations of miR-Rx34. Each drug was used at a concentration approximately equal to its IC₅₀ and at concentrations within 2-fold increments above or below. This matrix yielded a total of 49 different combinations representing 13 different ratios. Each drug was also used alone at these concentrations. Cells were reversed transfected with miR-Rx34 and incubated for 3 days until EMD1214063 was added to the medium. Another 3 days later, cellular proliferation was determined by AlamarBlue. Each data point was performed in triplicates, and the combination studies were repeated three times in each cell line.

### Data Analysis Methods

Combination index (CI) values were determined as described in the Synergy and combination index (CI) values section above.

Isobolograms: To describe the dose-dependent interaction of tivantinib and miR-Rx34, isobolograms at effect levels of 50% and 80% inhibition of cancer cell proliferation were created. Since the single agents—alone or in combination—usually reached 50% cancer cell inhibition, the 50% isobologram provided an actual comparison of the single use vs. the combination. The 80% isobologram was used to illustrate the utility of the combination at a high effect level that have practical implications in oncology. In each of these, additivity was determined by extrapolating the dose requirements for each drug in combination from its single use (IC50, IC80). Data points above or below the line of additivity indicate antagonism or synergy, respectively.

Curve shift analysis: To allow a direct comparison of the dose-response curves and to identify synergistic drug-drug interaction, non-linear regression trendlines of each drug alone or of the combination (IC50:IC50 ratio or other ratios where indicated) were normalized to its own IC50 value and referred to as IC50 equivalents (IC50 eq). IC50 equivalents of the combination were calculated according to

$${IC}_{50 eq} = \frac{{C}_{A , x}}{{IC}_{50 , A}} + \frac{{C}_{B , x}}{{IC}_{50 , B}} .$$

Data of the single agents and in combination were graphed in the same diagram to illustrate lower drug concentrations required to achieve any given effect relative to the single agents. This is represented in a left-shift of the dose-response curve and indicates synergy.

Statistical analysis: Statistical analysis was done using the Excel (Microsoft) and Graphpad software. Averages and standard deviations were calculated from triplicate experiments. Goodness of fit of non-linear regression trendlines was described by R2 values (Graphpad).

Results:

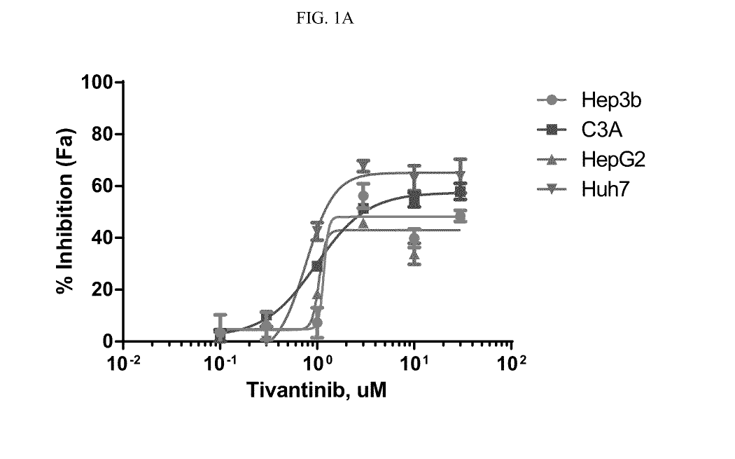

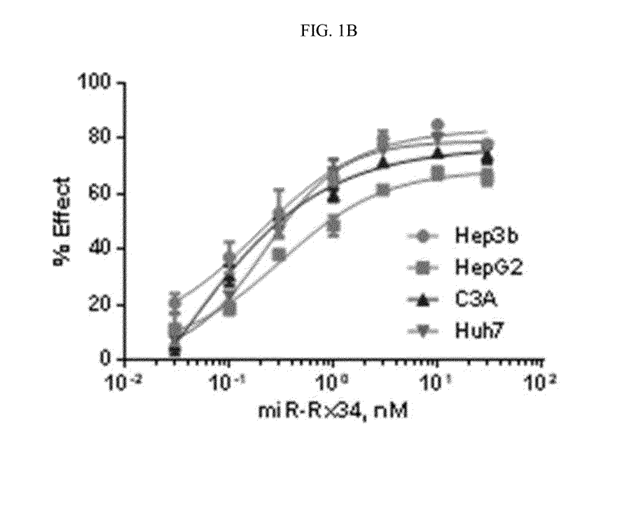

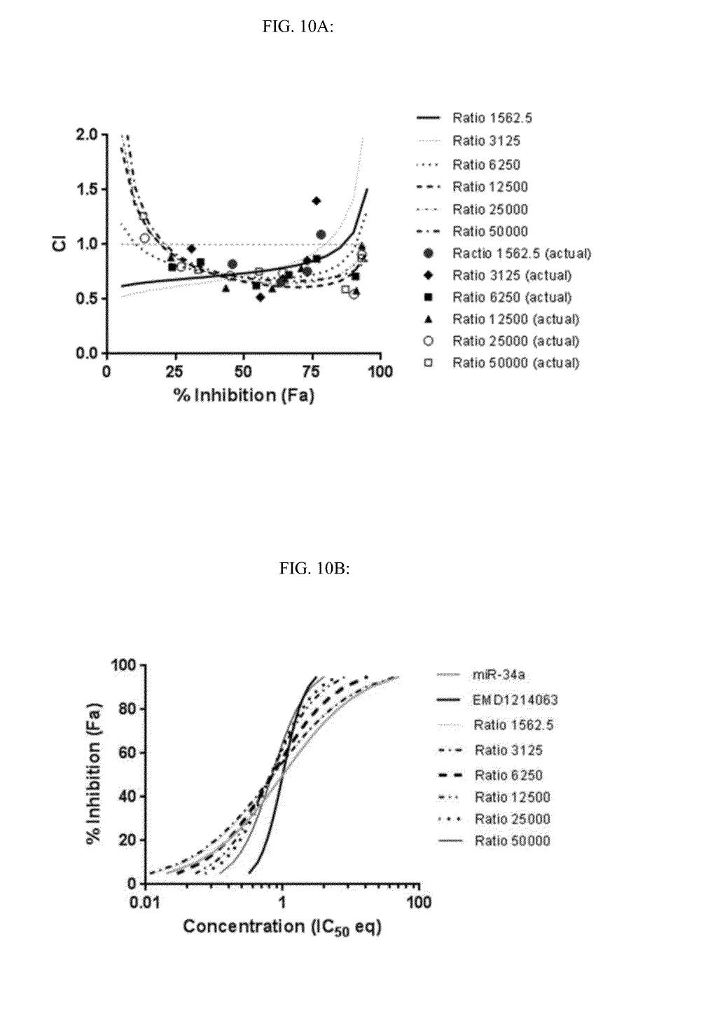

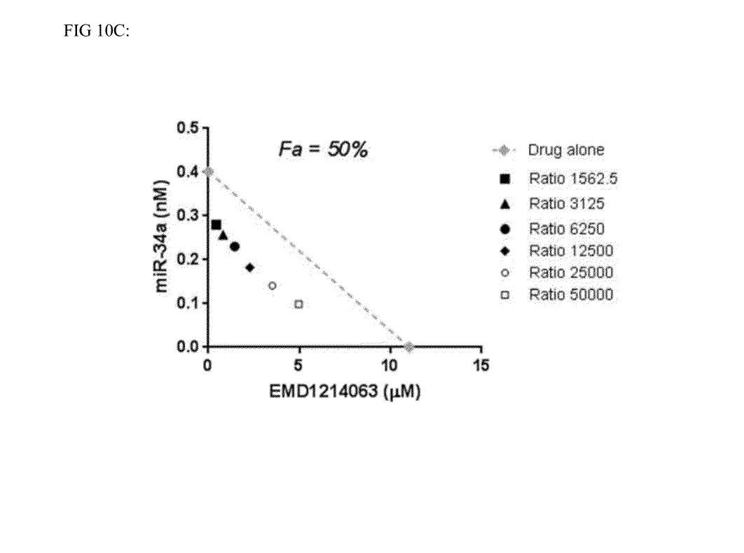

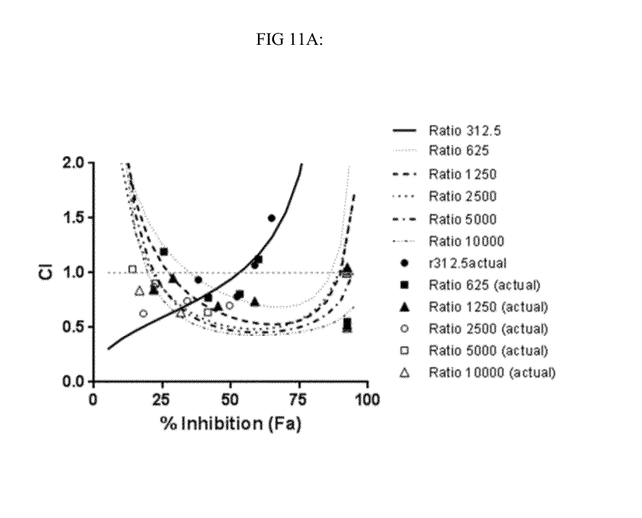

Table 3 and FIGS. 1-11 present the results of Example 1.

TABLE 3

| | 34a IC50, | Tivantinib | EMD1214063 | Fixed IC50 |
|---|---|---|---|---|
| Cell Line | nM * | IC50, μM | IC50, μM | ratio in combo |
| Hep3b | 1.1-7.2 | 4.63-18.6 | — | 3750 |
| HepG2 | 1.7-6 | 8.99-25 | — | 4000 |
| C3A | 0.2-0.6 | 2.3-7.42 | — | 12500 |
| Huh7 | 0.5-2 | 1.5-3.32 | — | 1500 |
| C3A | 0.5 | — | 7.0 | 3333.3 |
| SK-Hep1 | 3.5 | — | 6.7 | 333.3 |

* miR-Rx34 IC50 values in HCC cells are based on a previous study (data not shown).

FIGS. 1A-C present dose response curves of tivantinib alone (FIG. 1A), miR-34 alone (FIG. 1B), and EMD1214063 alone in HCC cells. Cells were treated with tivantinib alone at indicated concentrations, and cell proliferation was measured 3 days post drug treatment. Non-linear dose-response curves and IC50 values were calculated using GraphPad.

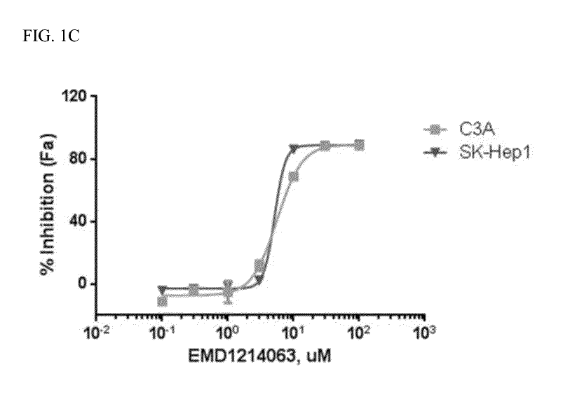

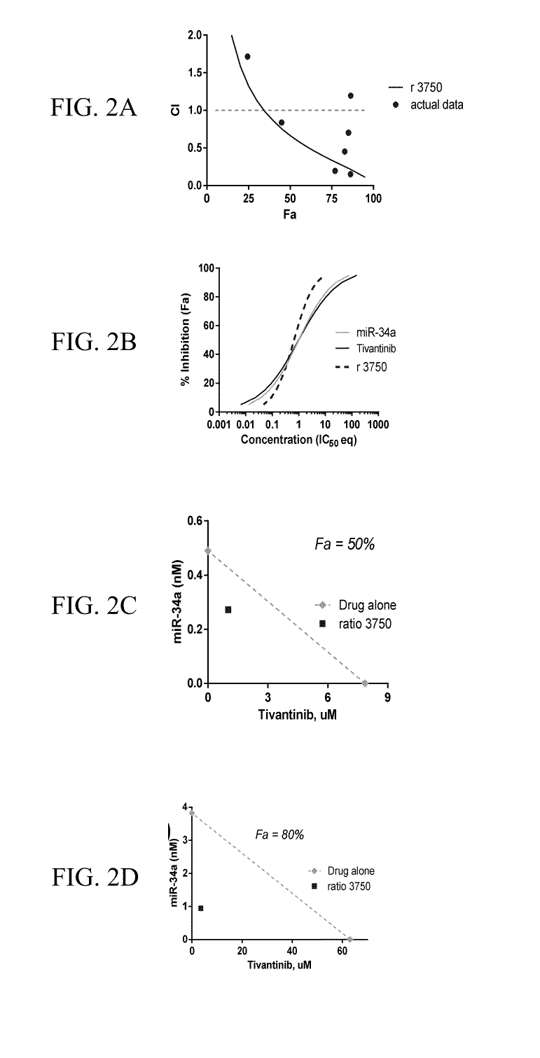

FIGS. 2A-D illustrates tivantinib and miR-Rx34 synergizing in Hep3B HCC cells. FIG. 2A illustrates combination index (CI) analysis. CI values were generated by non-linear regression methods. Trendlines indicate CI values at any given effect (Fa, fraction affected, % inhibition), and symbols represent CI values derived from actual data points. CI=1, additivity; CI>1, antagonism; CI<1, synergy. FIG. 2B illustrates curve shift analysis. Data derived from non-linear dose-response curves were normalized to IC50 values of the single agents (IC50 eq) and plotted in the same graph. Left and right shifts of the dose-response curves of the combination relative to the dose-response curves of the single agents indicate synergy or antagonism, respectively. FIGS. 2C and 2D illustrate isobologram analysis. The diagonal, dotted line indicates additivity, and the square symbol shows dose requirements to achieve 50% and 80% cancer cell inhibition, respectively. Data points below the line of additivity indicate synergy, data points above denote antagonism. Each data point is an average of raw data in triplicates, and the experiment has been repeated three times in each cell line.

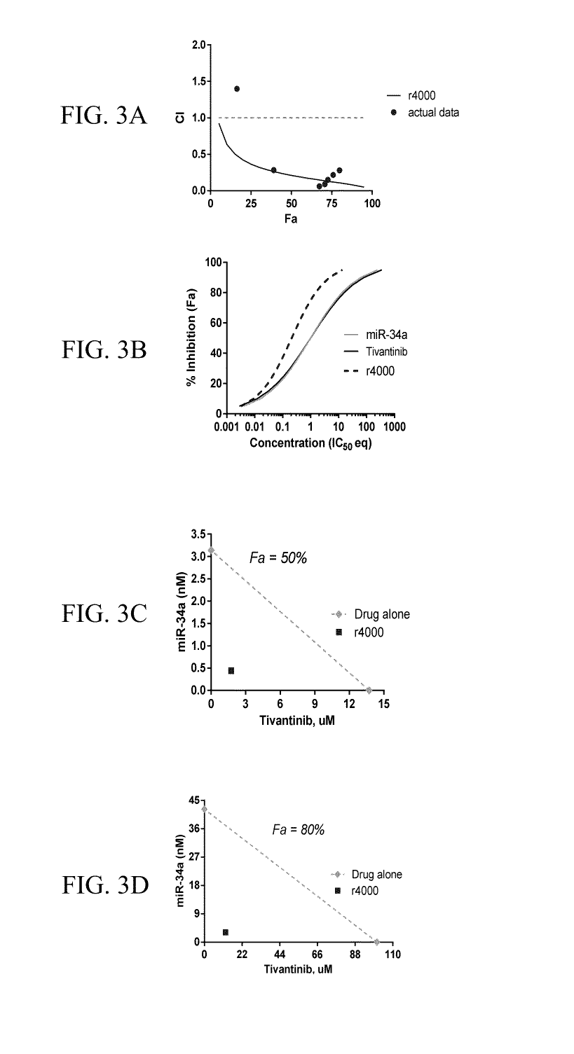

FIGS. 3A-D illustrates tivantinib and miR-Rx34 synergizing in HepG2 HCC cells. FIG. 3A illustrates combination index (CI) analysis. FIG. 3B illustrates curve shift analysis. FIGS. 3C and 3D illustrate isobologram analysis at dose requirements to achieve 50% and 80% cancer cell inhibition, respectively. The generation and analysis of this information is the same as described in connection with FIGS. 2A-D above.

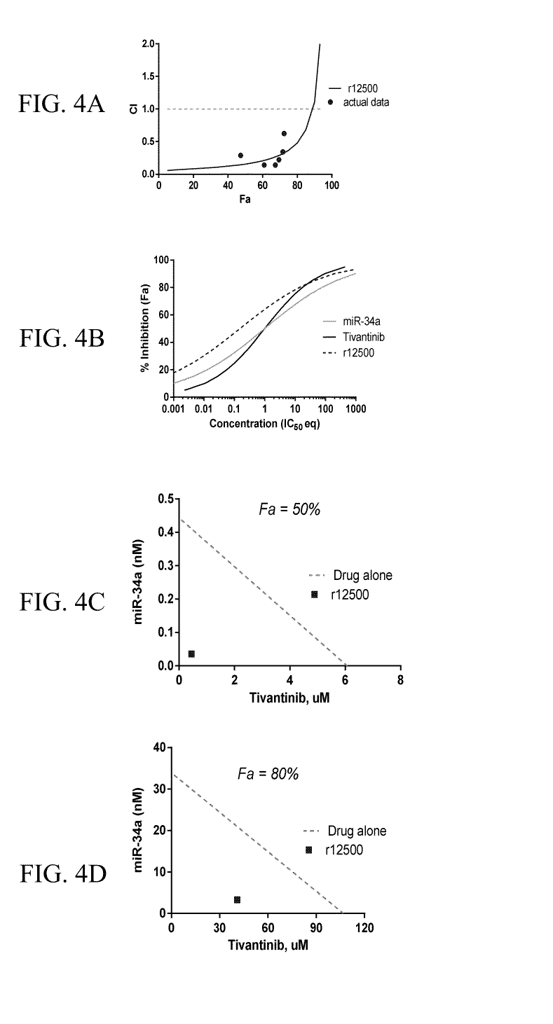

FIGS. 4A-D illustrates tivantinib and miR-Rx34 synergizing in C3A HCC cells. FIG. 4A illustrates combination index (CI) analysis. FIG. 4B illustrates curve shift analysis. FIGS. 4C and 4D illustrate isobologram analysis at dose requirements to achieve 50% and 80% cancer cell inhibition, respectively. The generation and analysis of this information is the same as described in connection with FIGS. 2A-D above.

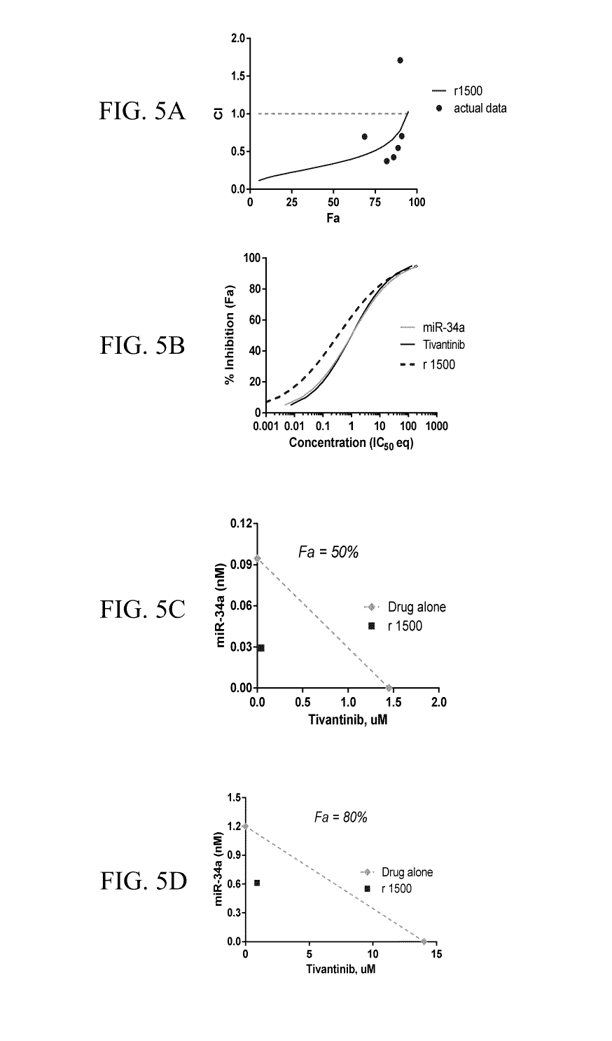

FIGS. 5A-D illustrates tivantinib and miR-Rx34 synergizing in Huh7 HCC cells. FIG. 5A illustrates combination index (CI) analysis. FIG. 5B illustrates curve shift analysis. FIGS. 5C and 5D illustrate isobologram analysis at dose requirements to achieve 50% and 80% cancer cell inhibition, respectively. The generation and analysis of this information is the same as described in connection with FIGS. 2A-D above.

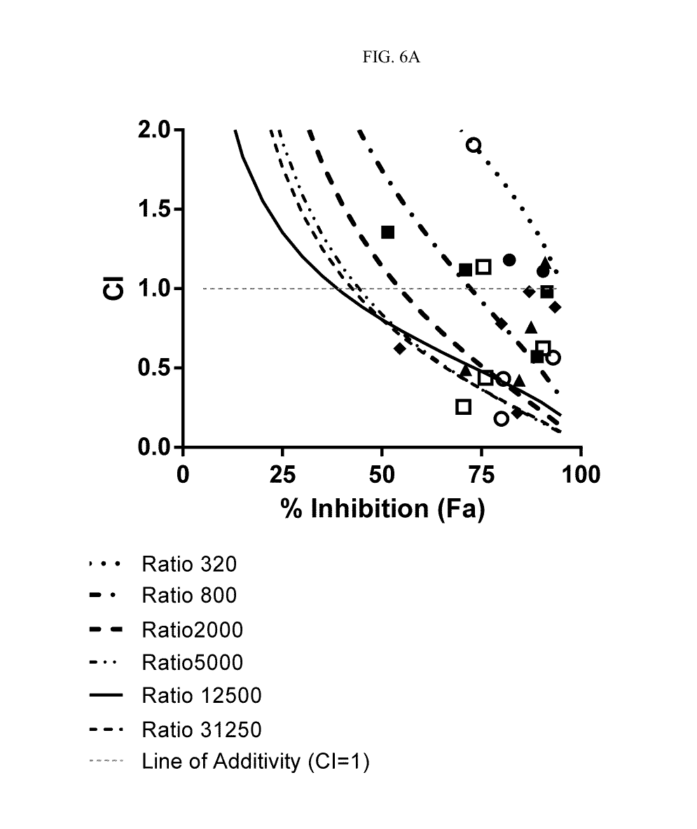

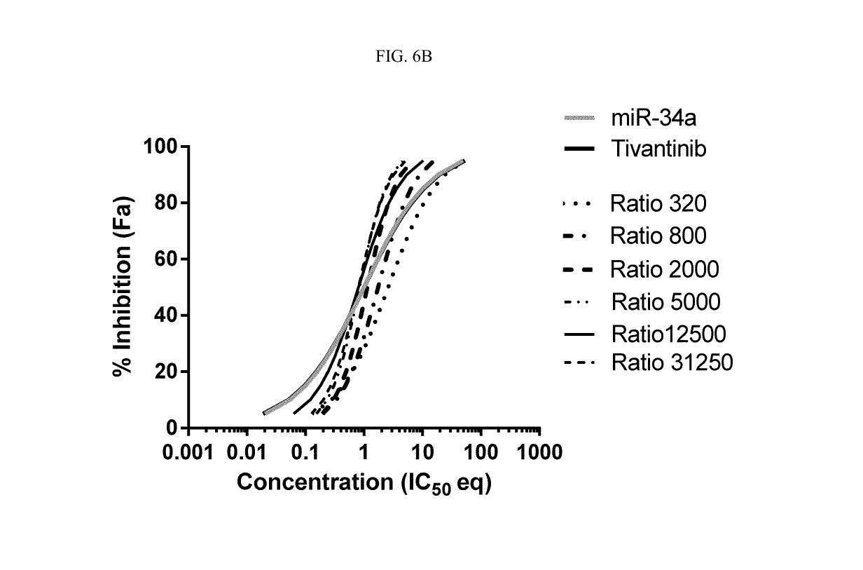

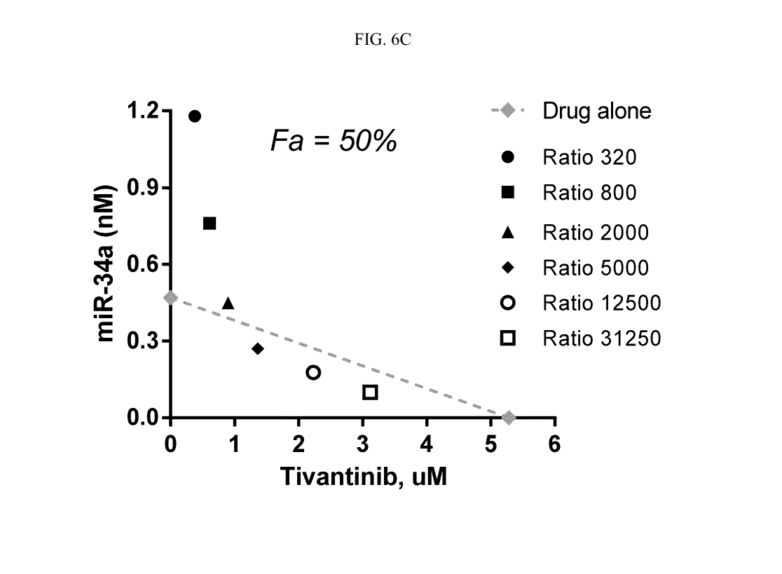

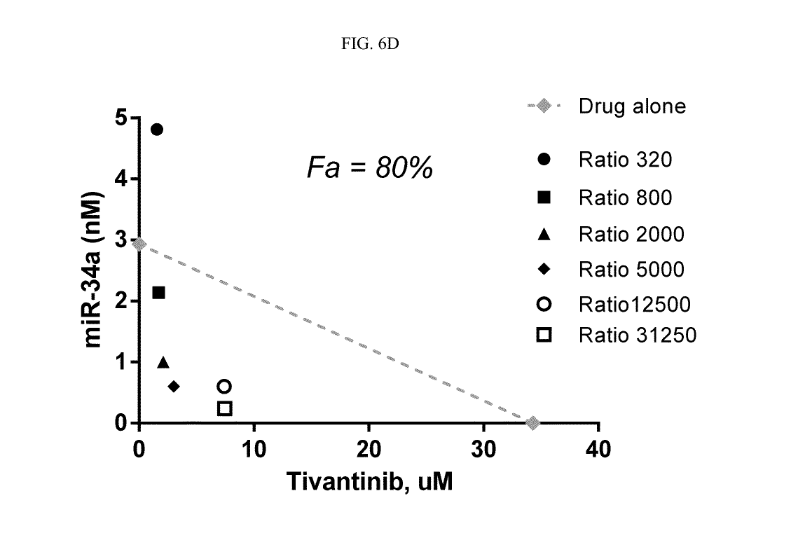

FIG. 6A-D illustrates synergistic effects between tivantinib and miR-Rx34 at multiple ratios in Hep3B cells. FIG. 6A illustrates combination index plot of various drug ratios. FIG. 6B illustrates curve shift analysis of various drug ratios. FIG. 6C illustrates isobologram at 50% and FIG. 6D illustrates isobologram at 80% cancer cell inhibition.

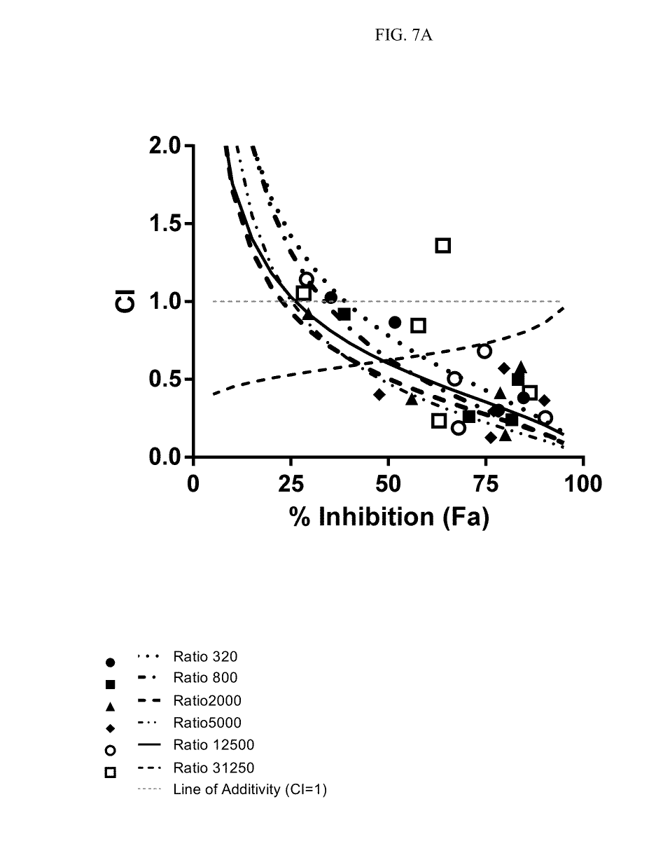

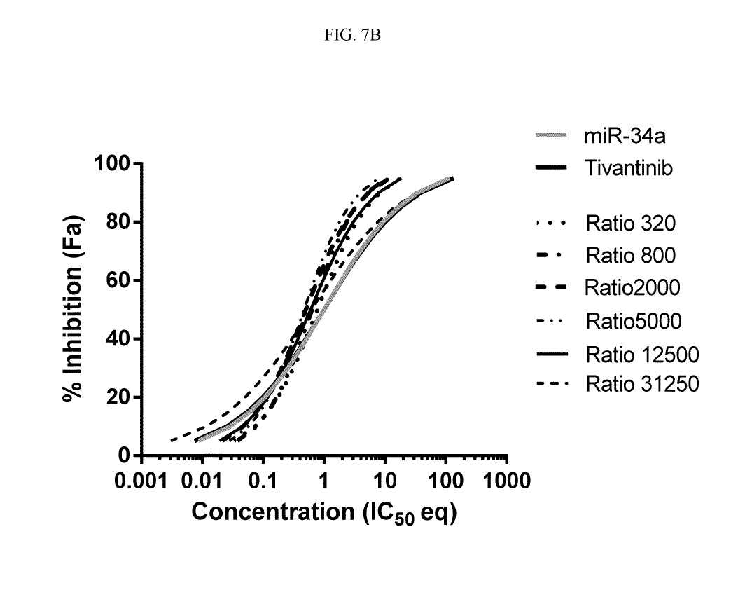

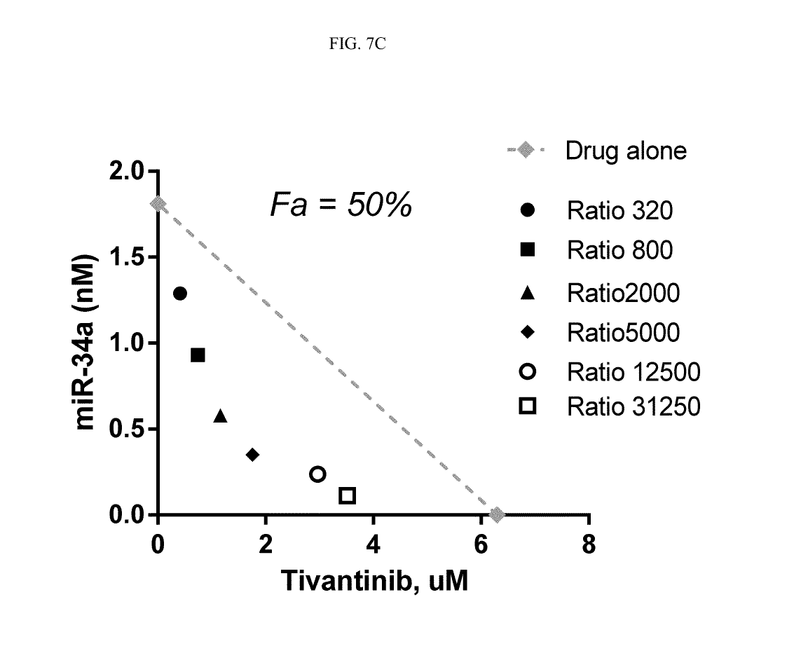

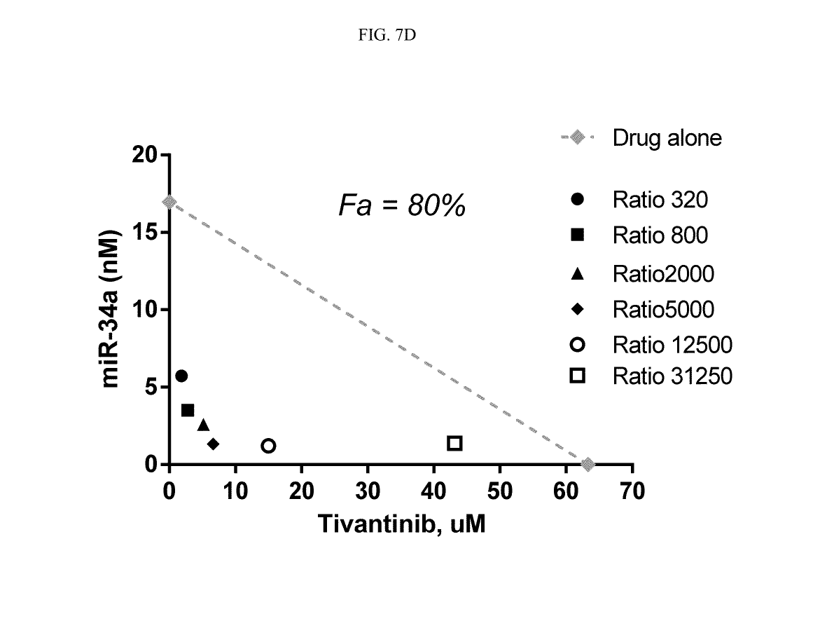

FIG. 7A-D illustrates synergistic effects between tivantinib and miR-Rx34 at multiple ratios in HepG2 cells. FIG. 7A illustrates combination index plot of various drug ratios. FIG. 7B illustrates curve shift analysis of various drug ratios. FIG. 7C illustrates isobologram at 50% and FIG. 7D illustrates isobologram at 80% cancer cell inhibition.

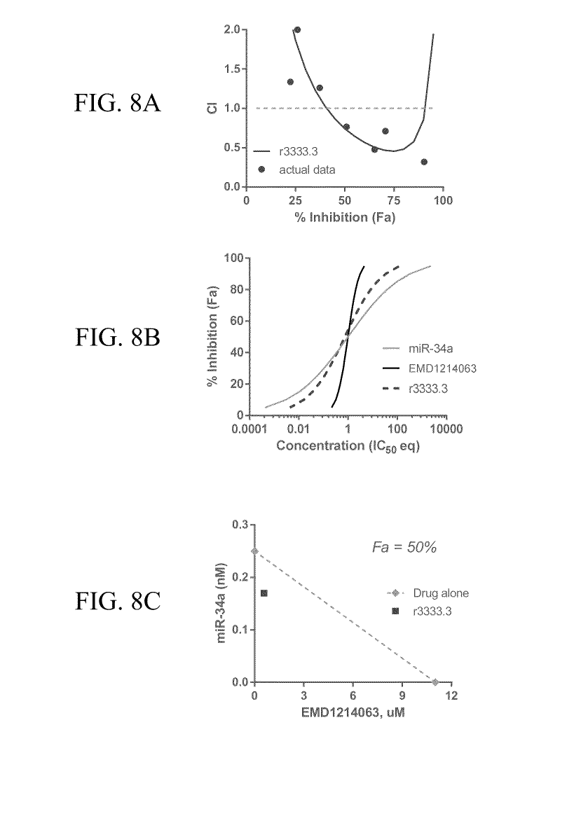

FIG. 8A-D illustrates EMD1214063 and miR-Rx34 synergizing in C3A HCC cells. FIG. 8A illustrates combination index (CI) analysis. FIG. 8B illustrates curve shift analysis. FIGS. 8C and 8D illustrate isobologram analysis at dose requirements to achieve 50% and 80% cancer cell inhibition, respectively. The generation and analysis of this information is the same as described in connection with FIGS. 2A-D above.

FIG. 8A-C illustrates EMD1214063 and miR-Rx34 synergizing in C3A HCC cells. FIG. 8A illustrates combination index (CI) analysis. FIG. 8B illustrates curve shift analysis. FIG. 8C illustrates isobologram analysis at dose requirements to achieve 50% cancer cell inhibition. The generation and analysis of this information is the same as described in connection with FIGS. 2A-C above.

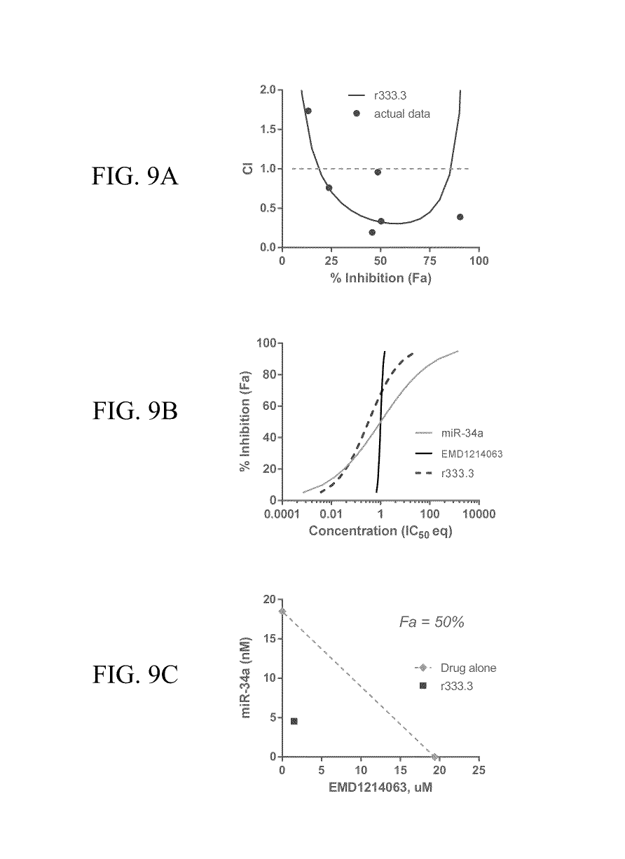

FIG. 9A-C illustrates EMD1214063 and miR-Rx34 synergizing in SK-Hep1 HCC cells. FIG. 9A illustrates combination index (CI) analysis. FIG. 9B illustrates curve shift analysis. FIG. 9C illustrates isobologram analysis at dose requirements to achieve 50% cancer cell inhibition. The generation and analysis of this information is the same as described in connection with FIGS. 2A-D above.

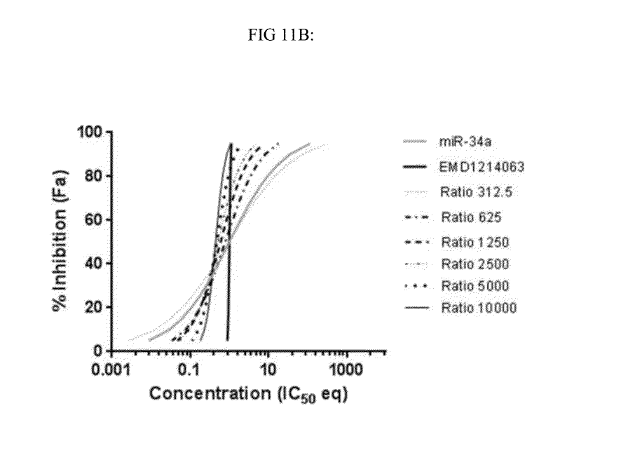

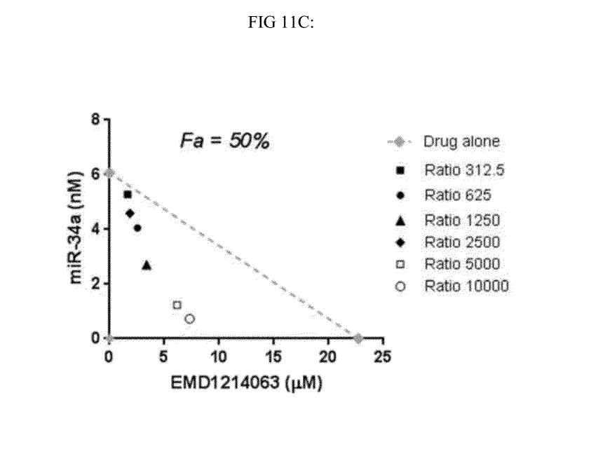

FIG. 11A-C illustrates synergistic effects between EMD1214063 and miR-Rx34 at multiple ratios in SK-Hep1 HCC cells. FIG. 11A illustrates combination index plot of various drug ratios. FIG. 11B illustrates curve shift analysis of various drug ratios. FIG. 11C illustrates isobologram at 50% cancer cell inhibition.

### CONCLUSIONS

Synergistic effects were observed between tivantinib and miR-Rx34 in all liver cancer cells tested. The data suggested that the combination between tivantinib and miR-Rx34 produced higher synergistic effects in HepG2 than in Hep3B cells at multiple ratios. Not all ratios produced the same synergistic effects, which suggest that optimizing combination chemotherapy can be controlled by drug ratios.

### Example 2

### Tivantinib:miR-Rx34 Dosing (Daily Ratios)

Tables 4-5 below illustrate examples of clinically relevant tivantinib:miR-Rx34 dosing ratios. The ratios are calculated over one day's dosing based upon (1) a 70 kg patient, (2) 120, 240, 480, or 720 mg BID tivantinib (twice daily), and (3) 20, 33, 50, 70, 93, 124, or 165 mg/m² miR-Rx34.

TABLE 4
Examples of tivantinib: miR-Rx34 dosing, with daily ratios ranging
from 15 to 764.

| mg ( based on 70 kg patient) | | | molar (based on 70 kg patient) | | | |
|---|---|---|---|---|---|---|
| | tivantinib | miR-Rx34 | miR-Rx34 | Molar | | |
| Ratio | [mg] | [mg] | [mg/m2] | Ratio | tivantinib | miR-Rx34 |
| 3171 | 120 | 38 | 20 | 127 | 0.3248 | 0.0026 |
| 1922 | 120 | 62 | 33 | 77 | 0.3248 | 0.0042 |
| 1269 | 120 | 95 | 50 | 51 | 0.3248 | 0.0064 |
| 906 | 120 | 132 | 70 | 36 | 0.3248 | 0.0089 |
| 682 | 120 | 176 | 93 | 27 | 0.3248 | 0.0119 |
| 512 | 120 | 235 | 124 | 21 | 0.3248 | 0.0158 |
| 384 | 120 | 312 | 165 | 15 | 0.3248 | 0.0210 |
| 6343 | 240 | 38 | 20 | 255 | 0.6497 | 0.0026 |
| 3844 | 240 | 62 | 33 | 154 | 0.6497 | 0.0042 |
| 2537 | 240 | 95 | 50 | 102 | 0.6497 | 0.0064 |
| 1812 | 240 | 132 | 70 | 73 | 0.6497 | 0.0089 |
| 1364 | 240 | 176 | 93 | 55 | 0.6497 | 0.0119 |
| 1023 | 240 | 235 | 124 | 41 | 0.6497 | 0.0158 |
| 769 | 240 | 312 | 165 | 31 | 0.6497 | 0.0210 |
| 12686 | 480 | 38 | 20 | 509 | 1.2993 | 0.0026 |
| 7688 | 480 | 62 | 33 | 309 | 1.2993 | 0.0042 |
| 5074 | 480 | 95 | 50 | 204 | 1.2993 | 0.0064 |
| 3624 | 480 | 132 | 70 | 146 | 1.2993 | 0.0089 |
| 2728 | 480 | 176 | 93 | 110 | 1.2993 | 0.0119 |
| 2046 | 480 | 235 | 124 | 82 | 1.2993 | 0.0158 |
| 1538 | 480 | 312 | 165 | 62 | 1.2993 | 0.0210 |
| 19029 | 720 | 38 | 20 | 764 | 1.9490 | 0.0026 |
| 11532 | 720 | 62 | 33 | 463 | 1.9490 | 0.0042 |
| 7611 | 720 | 95 | 50 | 306 | 1.9490 | 0.0064 |
| 5437 | 720 | 132 | 70 | 218 | 1.9490 | 0.0089 |
| 4092 | 720 | 176 | 93 | 164 | 1.9490 | 0.0119 |
| 3069 | 720 | 235 | 124 | 123 | 1.9490 | 0.0158 |
| 2306 | 720 | 312 | 165 | 93 | 1.9490 | 0.0210 |

Calculated using Tivantinib MW = 369.42 and miR-34a MW = 14834.

TABLE 5
Examples of tivantinib:synthetic miRNA molecule dosing, with daily
ratios ranging from 31-102 (1^{st} tier) and 62-204 (2^{nd} tier).

| 1st Tier Ratios | | | 2nd Tier Ratios | | |
|---|---|---|---|---|---|
| Ratio | tivantinib | miR-Rx34 | Ratio | tivantinib | miR-Rx34 |
| 102 | 0.649667 | 0.006377 | | | |
| 73 | 0.649667 | 0.008928 | | | |
| 55 | 0.649667 | 0.011861 | | | |
| 41 | 0.649667 | 0.015815 | | | |
| 31 | 0.649667 | 0.021044 | | | |
| | | | 204 | 1.299334 | 0.006377 |
| | | | 146 | 1.299334 | 0.008928 |
| | | | 110 | 1.299334 | 0.011861 |
| | | | 82 | 1.299334 | 0.015815 |
| | | | 62 | 1.299334 | 0.021044 |

### Example 3

### Tivantinib:miR-Rx34 Dosing (Weekly Ratios, Synthetic miRNA Molecule BIW)

Tables 6-7 below present examples of clinically relevant tivantinib:miR-Rx34 dosing ratios. The ratios are calculated over one week's dosing based upon (1) a 70 kg patient, (2) 120, 240, 480, or 720 mg BID tivantinib (twice daily), and (3) 20, 33, 50, 70, 93, 124, or 165 mg/m² miR-Rx34 give twice weekly.

TABLE 6
Examples of tivantinib:miR-Rx34 dosing, with weekly ratios ranging from 54 to 2674.

| Daily Dose | | | | | | | Weekly Dose | | |
|---|---|---|---|---|---|---|---|---|---|
| mg | | | | | | | All | | |
| (based on 70 kg patient) | | | | molar | | | Ratios | | |
| | | miR-Rx34 | miR-Rx34 | (based on 70 kg patient) | | | Molar | | |
| Ratio | Tiv. | [mg] | [mg/m2] | Ratio | Tiv. | miR-Rx34 | Ratios | Tiv. | miR-Rx34 |
| 3171 | 120 | 38 | 20 | 127 | 0.3248 | 0.0026 | 446 | 2.273835 | 0.005102 |
| 1922 | 120 | 62 | 33 | 77 | 0.3248 | 0.0042 | 270 | 2.273835 | 0.008417 |
| 1269 | 120 | 95 | 50 | 51 | 0.3248 | 0.0064 | 178 | 2.273835 | 0.012754 |
| 906 | 120 | 132 | 70 | 36 | 0.3248 | 0.0089 | 127 | 2.273835 | 0.017855 |
| 682 | 120 | 176 | 93 | 27 | 0.3248 | 0.0119 | 96 | 2.273835 | 0.023722 |
| 512 | 120 | 235 | 124 | 21 | 0.3248 | 0.0158 | 72 | 2.273835 | 0.031629 |
| 384 | 120 | 312 | 165 | 15 | 0.3248 | 0.0210 | 54 | 2.273835 | 0.042087 |
| 6343 | 240 | 38 | 20 | 255 | 0.6497 | 0.0026 | 891 | 4.547669 | 0.005102 |
| 3844 | 240 | 62 | 33 | 154 | 0.6497 | 0.0042 | 540 | 4.547669 | 0.008417 |
| 2537 | 240 | 95 | 50 | 102 | 0.6497 | 0.0064 | 357 | 4.547669 | 0.012754 |
| 1812 | 240 | 132 | 70 | 73 | 0.6497 | 0.0089 | 255 | 4.547669 | 0.017855 |
| 1364 | 240 | 176 | 93 | 55 | 0.6497 | 0.0119 | 192 | 4.547669 | 0.023722 |
| 1023 | 240 | 235 | 124 | 41 | 0.6497 | 0.0158 | 144 | 4.547669 | 0.031629 |
| 769 | 240 | 312 | 165 | 31 | 0.6497 | 0.0210 | 108 | 4.547669 | 0.042087 |
| 12686 | 480 | 38 | 20 | 509 | 1.2993 | 0.0026 | 1783 | 9.095339 | 0.005102 |
| 7688 | 480 | 62 | 33 | 309 | 1.2993 | 0.0042 | 1081 | 9.095339 | 0.008417 |
| 5074 | 480 | 95 | 50 | 204 | 1.2993 | 0.0064 | 713 | 9.095339 | 0.012754 |
| 3624 | 480 | 132 | 70 | 146 | 1.2993 | 0.0089 | 509 | 9.095339 | 0.017855 |
| 2728 | 480 | 176 | 93 | 110 | 1.2993 | 0.0119 | 383 | 9.095339 | 0.023722 |
| 2046 | 480 | 235 | 124 | 82 | 1.2993 | 0.0158 | 288 | 9.095339 | 0.031629 |
| 1538 | 480 | 312 | 165 | 62 | 1.2993 | 0.0210 | 216 | 9.095339 | 0.042087 |
| 19029 | 720 | 38 | 20 | 764 | 1.9490 | 0.0026 | 2674 | 13.64301 | 0.005102 |
| 11532 | 720 | 62 | 33 | 463 | 1.9490 | 0.0042 | 1621 | 13.64301 | 0.008417 |
| 7611 | 720 | 95 | 50 | 306 | 1.9490 | 0.0064 | 1070 | 13.64301 | 0.012754 |
| 5437 | 720 | 132 | 70 | 218 | 1.9490 | 0.0089 | 764 | 13.64301 | 0.017855 |
| 4092 | 720 | 176 | 93 | 164 | 1.9490 | 0.0119 | 575 | 13.64301 | 0.023722 |
| 3069 | 720 | 235 | 124 | 123 | 1.9490 | 0.0158 | 431 | 13.64301 | 0.031629 |
| 2306 | 720 | 312 | 165 | 93 | 1.9490 | 0.0210 | 327 | 13.64301 | 0.042087 |

Calculated using Tivantimb MW = 369.42 and miR-Rx34 MW = 14834.

TABLE 7
Examples of tivantinib:synthetic miRNA molecule dosing, with
weekly ratios ranging from 108-357 (1^{st} tier) and 216-713 (2^{nd} tier).

| 1st Tier Ratios | | | 2nd Tier Ratios | | |
|---|---|---|---|---|---|
| Ratio | tivantinib | miR-Rx34 | Ratio | tivantinib | miR-Rx34 |
| 357 | 4.547669 | 0.012754 | | | |
| 255 | 4.547669 | 0.017855 | | | |
| 192 | 4.547669 | 0.023722 | | | |
| 144 | 4.547669 | 0.031629 | | | |
| 108 | 4.547669 | 0.042087 | | | |
| | | | 713 | 9.095339 | 0.012754 |
| | | | 509 | 9.095339 | 0.017855 |
| | | | 383 | 9.095339 | 0.023722 |
| | | | 288 | 9.095339 | 0.031629 |
| | | | 216 | 9.095339 | 0.042087 |

### Example 4

### Tivantinib:miR-Rx34 Dosing (Weekly Ratios, Synthetic miRNA Molecule 5xQD)

Tables 8-9 below present examples of clinically relevant tivantinib: miR-Rx34 dosing ratios. The ratios are calculated over one week's dosing based upon (1) a 70 kg patient, (2) 120, 240, 480, or 720 mg BID tivantinib (twice daily), and (3) 20, 33, 50, 70, 93, 124, or 165 mg/m² miR-Rx34 given 5xQD (five consecutive days of a week).

TABLE 8
Examples of tivantinib:miR-Rx34 dosing, with daily ratios ranging from 22 to 1070.

| Daily Dose | | | | | | | Weekly Dose | | |
|---|---|---|---|---|---|---|---|---|---|
| mg | | | | | | | All | | |
| (based on 70 kg patient) | | | | molar | | | Ratios | | |
| | | miR-Rx34 | miR-Rx34 | (based on 70 kg patient) | | | Molar | | |
| Ratio | Tiv. | [mg] | [mg/m2] | Ratio | Tiv. | miR-Rx34 | Ratios | Tiv. | miR-Rx34 |
| 3171 | 120 | 38 | 20 | 127 | 0.3248 | 0.0026 | 178 | 2.273835 | 0.012754 |
| 1922 | 120 | 62 | 33 | 77 | 0.3248 | 0.0042 | 108 | 2.273835 | 0.021044 |
| 1269 | 120 | 95 | 50 | 51 | 0.3248 | 0.0064 | 71 | 2.273835 | 0.031884 |
| 906 | 120 | 132 | 70 | 36 | 0.3248 | 0.0089 | 51 | 2.273835 | 0.044638 |
| 682 | 120 | 176 | 93 | 27 | 0.3248 | 0.0119 | 38 | 2.273835 | 0.059305 |
| 512 | 120 | 235 | 124 | 21 | 0.3248 | 0.0158 | 29 | 2.273835 | 0.079073 |
| 384 | 120 | 312 | 165 | 15 | 0.3248 | 0.0210 | 22 | 2.273835 | 0.105218 |
| 6343 | 240 | 38 | 20 | 255 | 0.6497 | 0.0026 | 357 | 4.547669 | 0.012754 |
| 3844 | 240 | 62 | 33 | 154 | 0.6497 | 0.0042 | 216 | 4.547669 | 0.021044 |
| 2537 | 240 | 95 | 50 | 102 | 0.6497 | 0.0064 | 143 | 4.547669 | 0.031884 |
| 1812 | 240 | 132 | 70 | 73 | 0.6497 | 0.0089 | 102 | 4.547669 | 0.044638 |
| 1364 | 240 | 176 | 93 | 55 | 0.6497 | 0.0119 | 77 | 4.547669 | 0.059305 |
| 1023 | 240 | 235 | 124 | 41 | 0.6497 | 0.0158 | 58 | 4.547669 | 0.079073 |
| 769 | 240 | 312 | 165 | 31 | 0.6497 | 0.0210 | 43 | 4.547669 | 0.105218 |
| 12686 | 480 | 38 | 20 | 509 | 1.2993 | 0.0026 | 713 | 9.095339 | 0.012754 |
| 7688 | 480 | 62 | 33 | 309 | 1.2993 | 0.0042 | 432 | 9.095339 | 0.021044 |
| 5074 | 480 | 95 | 50 | 204 | 1.2993 | 0.0064 | 285 | 9.095339 | 0.031884 |
| 3624 | 480 | 132 | 70 | 146 | 1.2993 | 0.0089 | 204 | 9.095339 | 0.044638 |
| 2728 | 480 | 176 | 93 | 110 | 1.2993 | 0.0119 | 153 | 9.095339 | 0.059305 |
| 2046 | 480 | 235 | 124 | 82 | 1.2993 | 0.0158 | 115 | 9.095339 | 0.079073 |
| 1538 | 480 | 312 | 165 | 62 | 1.2993 | 0.0210 | 86 | 9.095339 | 0.105218 |
| 19029 | 720 | 38 | 20 | 764 | 1.9490 | 0.0026 | 1070 | 13.64301 | 0.012754 |
| 11532 | 720 | 62 | 33 | 463 | 1.9490 | 0.0042 | 648 | 13.64301 | 0.021044 |
| 7611 | 720 | 95 | 50 | 306 | 1.9490 | 0.0064 | 428 | 13.64301 | 0.031884 |
| 5437 | 720 | 132 | 70 | 218 | 1.9490 | 0.0089 | 306 | 13.64301 | 0.044638 |
| 4092 | 720 | 176 | 93 | 164 | 1.9490 | 0.0119 | 230 | 13.64301 | 0.059305 |
| 3069 | 720 | 235 | 124 | 123 | 1.9490 | 0.0158 | 173 | 13.64301 | 0.079073 |
| 2306 | 720 | 312 | 165 | 93 | 1.9490 | 0.0210 | 130 | 13.64301 | 0.105218 |

Calculated using Tivantmib MW = 369.42 and miR-Rx34 MW = 14834.

TABLE 9
Examples of tivantinib:miR-Rx34 dosing, with weekly ratios
ranging from 43-143 (1^{st} tier) and 86-285 (2^{nd} tier).

| 1st Tier Ratios | | | 2nd Tier Ratios | | |
|---|---|---|---|---|---|
| Ratio | tivantinib | miR-Rx34 | Ratio | tivantinib | miR-Rx34 |
| 143 | 4.547669 | 0.031884 | | | |
| 102 | 4.547669 | 0.044638 | | | |
| 77 | 4.547669 | 0.059305 | | | |
| 58 | 4.547669 | 0.079073 | | | |
| 43 | 4.547669 | 0.105218 | | | |
| | | | 285 | 9.095339 | 0.031884 |
| | | | 204 | 9.095339 | 0.044638 |
| | | | 153 | 9.095339 | 0.059305 |
| | | | 115 | 9.095339 | 0.079073 |
| | | | 86 | 9.095339 | 0.105218 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating a cancer in an individual in need thereof, comprising:
administering to the individual:
(a) c-Met inhibitor; and
(b) a synthetic miRNA molecule, comprising:
(i) an active strand comprising a sequence according to SEQ ID NO:9; and
(ii) a passenger strand that is at least 60% complementary to the active strand, wherein the passenger strand comprises a 5′ terminal cap.

2. (canceled)

3. The method of claim 1, wherein the 5′ terminal cap is a lower alkylamine.

4. The method of claim 1, wherein the 5′ terminal cap is NH₂—(CH₂)₆—O—.

5. The method of claim 1, wherein the active strand is at least 80% identical to a sequence encoding mature miR-34a (SEQ ID NO: 1).

6. The method of claim 1, wherein the active strand is at least 80% identical to a sequence encoding mature miR-34b (SEQ ID NO: 2).

7. The method of claim 1, wherein the active strand is at least 80% identical to a sequence encoding mature miR-34c (SEQ ID NO: 3).

8. The method of claim 1, wherein the active strand comprises a sequence according to SEQ ID NO:4.

9. The method of claim 1, wherein the active strand is at least 80% identical to a sequence encoding mature miR-449a (SEQ ID NO: 5).

10. The method of claim 1, wherein the active strand is at least 80% identical to a sequence encoding mature miR-449b (SEQ ID NO: 6).

11. The method of claim 1, wherein the active strand is at least 80% identical to a sequence encoding mature miR-449c (SEQ ID NO: 7).

12. The method of claim 1, wherein the active strand comprises a sequence according to SEQ ID NO: 8.

13. (canceled)

14. The method of claim 1, wherein the cancer is pancreatic, gastric, lung, thyroid, brain, kidney, head and neck, or liver cancer.

15. The method of claim 1, wherein the cancer is liver cancer.

16. The method of claim 15, wherein the liver cancer is primary liver cancer.

17. The method of claim 15, wherein the liver cancer is hepatocellular carcinoma (HCC).

18.-21. (canceled)

22. The method of claim 1, wherein the c-Met inhibitor is selected from the group consisting of: ARQ197 (Tivantinib), GSK/1363089/XL880 (Foretinib), XL184 (Cabozantinib), HMPL-504/AZD6094/volitinib (Savolitinib), MSC2156119J (EMD 1214063, Tepotinib), LY2801653 (Merestinib), AMG 337, INCB28060 (Capmatinib), AMG 458, PF-04217903, PF-02341066 (Crizotinib), E7050 (Golvatinib), MK-2461, BMS-777607, JNJ-38877605, EMD1214063 (MSC2156119J; Tepotinib), SOMG-833, or pharmaceutically acceptable salts thereof.

23. (canceled)

24. The method of claim 1, wherein the c-Met inhibitor is ARQ197 (tivantinib).

25. The method of claim 1, wherein the c-Met inhibitor is EMD1214063 (MSC2156119J; Tepotinib).

26-35. (canceled)

36. The method of claim 1, wherein the cancer has primary resistance to the c-Met inhibitor.

37. The method of claim 1, wherein the cancer has secondary resistance to the c-Met inhibitor.

38-128. (canceled)

129. The method of claim 1, wherein the synthetic miRNA molecule is administered in a liposomal formulation.

130-136. (canceled)

137. The method of claim 1, further comprising administering dexamethasone to the individual.

138. (canceled)

139. The method of claim 1, further comprising identifying the cancer as having resistance to a c-Met inhibitor.


SEQUENCE LISTING (9 sequences)

SEQ ID NO: 1 - RNA, 23 nt, Artificial Sequence
  Description of Artificial Sequence Synthetic oligonucleotide
uggcagugucuuagcugguuguu

SEQ ID NO: 2 - RNA, 23 nt, Artificial Sequence
  Description of Artificial Sequence Synthetic oligonucleotide
uaggcagugucauuagcugauug

SEQ ID NO: 3 - RNA, 23 nt, Artificial Sequence
  Description of Artificial Sequence Synthetic oligonucleotide
aggcaguguaguuagcugauugc

SEQ ID NO: 4 - RNA, 18 nt, Artificial Sequence
  Description of Artificial Sequence Synthetic oligonucleotide
ggcaguguuuagcuguug

SEQ ID NO: 5 - RNA, 22 nt, Artificial Sequence
  Description of Artificial Sequence Synthetic oligonucleotide
uggcaguguauuguuagcuggu

SEQ ID NO: 6 - RNA, 22 nt, Artificial Sequence
  Description of Artificial Sequence Synthetic oligonucleotide
aggcaguguauuguuagcuggc

SEQ ID NO: 7 - RNA, 25 nt, Artificial Sequence
  Description of Artificial Sequence Synthetic oligonucleotide
uaggcaguguauugcuagcggcugu

SEQ ID NO: 8 - RNA, 19 nt, Artificial Sequence
  Description of Artificial Sequence Synthetic oligonucleotide
uggcaguguauuguagcgg

SEQ ID NO: 9 - RNA, 7 nt, Artificial Sequence
  Description of Artificial Sequence Synthetic oligonucleotide
ggcagug